(19) United States
(12) Patent Application Publication
(10) Pub. No.: US 2024/0226184 A1
(43) Pub. Date: Jul. 11, 2024

(54) INSECT PROTEIN COMPOSITION FOR THE PREVENTION AND TREATMENT OF OSTEARTHRITIS

(71) Applicant: Protix B.V., Dongen (NL)

(72) Inventors: Aman Paul, Dongen (NL); Nuria Martin Tomé, Dongen (NL); Arpita Chakraborty, Dongen (NL)

(73) Assignee: Protix B.V., Dongen (NL)

(21) Appl. No.: 18/563,881

(22) PCT Filed: May 2, 2022
(86) PCT No.: PCT/NL2022/050235

§ 371 Date: Nov. 23, 2023

(30) Foreign Application Priority Data
May 28, 2021 (NL) 2028315

(51) Int. Cl. A61K 35/64; A23K 20/142; A23L 33/18; A61K 38/01

(52) U.S. Cl. CPC A61K 35/64; A23K 20/142; A23L 33/18; A61K 38/01

(57) ABSTRACT

The invention relates to an insect protein composition for use as a medicament, or for use in a method for the prophylaxis or treatment of inflammation, osteoarthritis, synovial inflammation, synovial macrophage activation, macrophage-induced synovial damage, or low-grade inflammatory disease of a joint. Typically, the insect is black soldier fly larvae. Typically, the protein is water-soluble protein and/or enzymatically hydrolysed protein. Preferably, the composition comprises glucosamine. As part of the invention, the insect protein composition inhibits and/or prevents macrophage activation, and/or prevents or inhibits macrophage-induced synovial damage, and/or inhibits and/or prevents cell lysis and/or inhibits and/or prevents reactive oxygen species formation by a cell and/or comprises glucosamine. The invention also relates to a food or feed supplement, ingredient or product, comprising the insect protein composition. Furthermore, the invention relates to a non-therapeutic method of prevention against, providing relief from or amelioration of a joint problem in a human subject or an animal such as a horse, a pet such as a dog or a cat, preferably a human subject or a dog, the method comprising orally administering to the human subject or the animal the insect protein composition. The invention also relates to a pharmaceutical composition comprising the insect protein composition, and to the pharmaceutical composition for use as a medicament and for use in the treatment or prevention of osteoarthritis.

### TECHNICAL FIELD

The invention relates to an insect protein composition for use as a food supplement or a feed supplement, or a medicament, or to an insect protein composition for use in a method for the prophylaxis or treatment of inflammation, osteoarthritis, synovial inflammation, synovial macrophage activation or low-grade inflammatory disease of a joint. Typically, the insect is black soldier fly larvae. Typically, the protein is water-soluble protein such as a water-soluble protein fraction from insect, and/or enzymatically hydrolysed protein, such as enzymatically hydrolysed insect protein. As part of the invention, the insect protein composition inhibits and/or prevents oxidative damage induced by macrophages and/or inhibits and/or prevents macrophage activation and/or inhibits and/or prevents cell lysis and/or inhibits and/or prevents reactive oxygen species formation by a cell and/or comprises glucosamine. The invention also relates to a food or feed supplement, ingredient or product, comprising the insect protein composition. Furthermore, the invention relates to a non-therapeutic method of prevention against, providing relief from or amelioration of a joint problem in a human subject or an animal such as a horse, a pet such as a dog or a cat, preferably a human subject or a dog, the method comprising orally administering to the human subject or the animal the insect protein composition. The invention also relates to a pharmaceutical composition comprising the insect protein composition, and to the pharmaceutical composition for use as a medicament and for use in the treatment or prevention of osteoarthritis.

### BACKGROUND

Osteoarthritis (OA) is one of the common pathologies affecting mammals such as ageing mammals, for example human subjects or animals such as pets, e.g. ageing dogs as well as cats. OA is a multifactorial and polygenic disease. OA affects the knee, hip, shoulder and/or elbow of the mammals such as human subjects and animals such as horses and dogs. OA initiation and progression is associated with several factors. Ageing related stress could result in the secretion of molecules such as chemokines, cytokines, proteases, etc. These molecules directly react with, or trigger production of compounds that react with, cellular components leading to degradation or structural changes in synovial membrane, articular cartilage and subchondral bone. As a result of these processes mammals such as human patients and animals may develop impaired joint functioning as well as painful locomotion. Current treatment of OA may involve use of nonsteroidal anti-inflammatory drugs such as acetaminophen which is often associated with several side effects. Veterinary professionals emphasize on the preventive approach by encouraging the use of natural pet food ingredients that could pro-actively prevent the formation of OA. That is to say, currently, treatments are directed to relief of symptoms such as pain, decreased mobility, etc., rather than to true curing the OA. Moreover, in view of the side-effects that are apparent when human patients or e.g. pets such as dogs are treated with currently available drugs, a solution still needs to be found that allows for preventing and/or curing OA and its disease symptoms (phenotype).

### SUMMARY

The inventors demonstrated the ability of black soldier fly larvae (BSF) protein compositions to protect the animal cells against the neutrophil mediated oxidative damage. Without wishing to be bound by any theory, it appears that due to the ability of BSF protein derivatives and BSF protein compositions to donate hydrogen atoms and/or electrons to counterpoise unstable molecules, such protein compositions are of use in prevention of e.g. arthritis formation. The inventors evaluated and established the role of BSF protein derivatives and BSF protein compositions in amongst others arthritis prevention as well as in for example treatment of arthritis, in particular osteoarthritis.

An aspect of the invention relates to an insect protein composition for use as a medicament.

An aspect of the invention relates to an insect protein composition for use in a method for the prophylaxis or treatment of inflammation.

An aspect of the invention relates to an insect protein composition for use in a method for the prophylaxis or treatment of inflammation in a joint, such as in a joint affected by osteoarthritis.

An aspect of the invention relates to an insect protein composition for use in a method for the prophylaxis or treatment of synovial inflammation and/or synovitis, such as in a joint affected by osteoarthritis.

An aspect of the invention relates to an insect protein composition for use in a method for the prophylaxis or treatment of damage of structural biomolecules of a joint, such as in a joint affected by osteoarthritis.

An aspect of the invention relates to an insect protein composition for use in a method for the prophylaxis or treatment of chondrocyte apoptosis, such as in a joint affected by osteoarthritis.

An aspect of the invention relates to an insect protein composition for use in a method for the prophylaxis or treatment of dysfunction of a subchondral bone and/or of subchondral bone remodeling and/or of bone loss, such as of a bone of a joint affected by osteoarthritis.

An aspect of the invention relates to an insect protein composition for use in a method for the prophylaxis or treatment of cartilage degradation, such as of cartilage of a joint affected by osteoarthritis. An aspect of the invention relates to an insect protein composition for use in a method for the prophylaxis or treatment of osteophyte formation, such as in a joint affected by osteoarthritis.

An aspect of the invention relates to an insect protein composition for use in a method for the prophylaxis or treatment of loss of articular cartilage, such as in a joint affected by osteoarthritis.

An aspect of the invention relates to an insect protein composition for use in a method for the prophylaxis or treatment of sclerosis of subchondral bone, such as a bone of a joint affected by osteoarthritis.

An aspect of the invention relates to an insect protein composition for use in a method for the prophylaxis or treatment of subchondral cysts formation, such as in a joint affected by osteoarthritis.

An aspect of the invention relates to an insect protein composition for use in a method for the prophylaxis or treatment of synovial damage such as induced by activated macrophages and/or of synovial macrophage activation, such as in synovium of a joint affected by osteoarthritis.

An aspect of the invention relates to an insect protein composition for use in a method for the prophylaxis or treatment of synovial macrophage activation and/or the prevention or treatment of macrophage-induced synovial damage, such as in synovium of a joint affected by osteoarthritis.

An aspect of the invention relates to an insect protein composition for use in a method for the prophylaxis or treatment of low-grade inflammatory disease of a joint, such as of a joint affected by osteoarthritis.

An aspect of the invention relates to an insect protein composition for use in a method for the prophylaxis or treatment of joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis.

An aspect of the invention relates to an insect protein composition for use in a method for the prophylaxis or treatment of innate immune response in a joint, such as in a joint affected by osteoarthritis.

An aspect of the invention relates to an insect protein composition for use in a method for the prophylaxis or treatment of joint stiffness and/or locomotor restriction and/or flexion contracture and/or decreased flexion, such as of a joint affected by osteoarthritis.

An aspect of the invention relates to an insect protein composition for use in a method for the prophylaxis or treatment of synovial fibrosis, such as in a joint affected by osteoarthritis.

An aspect of the invention relates to an insect protein composition for use in a method for the prophylaxis or treatment of joint-space narrowing, such as in a joint affected by osteoarthritis.

An aspect of the invention relates to an insect protein composition for use in a method for the prophylaxis or treatment of deterioration of articular cartilage and underlying bone, such as in a joint affected by osteoarthritis.

An aspect of the invention relates to an insect protein composition for use in a method for the prophylaxis or treatment of erythrocyte lysis and/or elevated erythrocyte sedimentation compared to normal, such as erythrocyte lysis and/or elevated erythrocyte sedimentation in synovium of a joint affected by osteoarthritis.

An aspect of the invention relates to an insect protein composition for use in a method for the prophylaxis or treatment of arthritis.

An aspect of the invention relates to an insect protein composition for use in a method for the prophylaxis or treatment of osteoarthritis.

An aspect of the invention relates to an insect protein composition for use in a method for the prophylaxis or treatment of osteoarthritis progression.

An aspect of the invention relates to an insect protein composition for use in a method for the prophylaxis or treatment of erosive osteoarthritis or inflammatory osteoarthritis.

An aspect of the invention relates to an insect protein composition for use in a method for the prophylaxis or treatment of early-stage osteoarthritis or late-stage osteoarthritis.

Preferred is the insect protein composition for use according to the invention, wherein the insect is larvae of black soldier fly.

An aspect of the invention relates to a human food supplement or animal feed supplement, human food ingredient or animal feed ingredient or human food product or animal feed product, comprising the insect protein composition of the invention. Optionally, the insect protein composition, e.g. protein composition derived from black soldier fly larvae, comprise glucosamine. If present in the insect protein composition, the amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

An aspect of the invention relates to use of the insect protein composition according to the invention in the preparation of a human food supplement or animal feed supplement, human food ingredient or animal feed ingredient or human food product or animal feed product. Optionally, the insect protein composition, e.g. protein composition derived from black soldier fly larvae, comprises glucosamine. If present in the insect protein composition, the amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

An aspect of the invention relates to a non-therapeutic method of prevention against, providing relief from or amelioration of a joint problem in a human subject or an animal such as a horse, a pet such as a dog or a cat, preferably a human subject or a dog, the method comprising orally administering to the human subject or the animal the insect protein composition of the invention or the human food supplement or animal feed supplement, human food ingredient or animal feed ingredient or human food product or animal feed product of the invention, wherein the joint problem is any one or more of: inflammation, for example inflammation in a joint affected by osteoarthritis, synovial inflammation and/or synovitis, such as in a joint affected by osteoarthritis, damage of structural biomolecules of the joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis. The human subject is a healthy human subject not suffering from OA, or is a patient with OA; the animal, for example a horse or a dog, preferably a dog, does not suffer from OA, or suffers from OA. Optionally, the insect protein composition, e.g. protein composition derived from black soldier fly larvae, comprises glucosamine. If present in the insect protein composition, the amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

An aspect of the invention relates to a pharmaceutical composition comprising the insect protein composition of the invention and optionally a pharmaceutically acceptable diluent and/or a pharmaceutically acceptable excipient. Optionally, said insect protein composition, e.g. protein composition derived from black soldier fly larvae, comprises glucosamine. If present in the insect protein composition, the amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

An aspect of the invention relates to the pharmaceutical composition of the invention for use according to the invention.

Preferred is the pharmaceutical composition for use of the invention in a method of treatment or prophylaxis of any one or more of osteoarthritis, osteoarthritis progression, erosive osteoarthritis, inflammatory osteoarthritis, early-stage osteoarthritis or late-stage osteoarthritis, wherein a human subject is treated or an animal such as a horse or a pet, for example a dog or a cat, preferably a human patient or a dog, such as a human patient or a dog suffering from osteoarthritis.

The present invention will be described with respect to particular embodiments but the invention is not limited thereto but only by the claims. The embodiments of the invention described herein can operate in combination and cooperation, unless specified otherwise. Moreover, the features of the invention described herein, e.g. features outlined in the context of certain aspects and embodiments, can operate in combination and cooperation, unless specified otherwise.

### Definitions

The term “insect” as used herein refers to an insect in any development stage, such as adult insects, insect larvae, insect prepupae and pupae.

The term “fresh insects” as used herein has its conventional meaning and refers to living insects or insects that have been killed shortly before being provided in step a) of the method of the invention, such as killed within 1 minute-3 hours before being subjected to step a). Fresh insects for example differ from stored insects that have been killed at a certain moment and stored at for example room temperature, 0° C.-8° C. or at a temperature below 0° C. for over 3 hours such as for days to weeks to months to years, before such stored insects are processed. Typically, fresh insects are living black soldier fly larvae.

The term “hatching” as used herein has its conventional meaning and refers to the process of young larvae emerging from the egg.

The term “larvae” as used herein has its conventional meaning and refers to the juvenile stadium of holometabolous insects, such as black soldier flies larvae.

The term “hatchling” or “neonate” as used herein has its conventional meaning and refers to larvae that have just hatched from the eggs.

The term “prepupae” as used herein refers to the last larval stage wherein the chitin content of the larvae has increased significantly.

The term “pupae” as used herein has its conventional meaning and refers to the stage of the insects life wherein the metamorphosis from larva to adult insect, such as black soldier flies

The term “pulp” or “insect pulp” or “puree” or “larvae puree” or “insect puree” or “minced BSF larvae” or the like as used herein all have the same meaning and refers to the product obtained after mechanical size reduction of the insects, such as by mincing, to a size of less than 0.5 mm.

The term “nutrient stream” as used herein has its conventional meaning and refers to streams that contain nutrients, such as fats, protein and protein-derived material, carbohydrates, minerals and/or chitin. Within the context of the present invention, chitin is also considered a nutrient. Within the context of the present invention, the insect puree or insect pulp obtained with the method of the invention is also considered a nutrient.

The term “anti-oxidant property” has its regular scientific meaning and here refers to a compound or a composition, such as the puree and the hydrolysed puree of the invention and obtainable with the method of the invention, that consists of or comprises an antioxidant with antioxidant activity, such as an anti-inflammatory response. Typically, such an anti-inflammatory response is a response to the inflammatory response induced by for example reactive oxygen species, e.g. in the cells of a mammal such as a pet or a human subject, or in the cells of a fish. Reference is made to for example book chapters 1, 5 and 6 of “Antioxidants in Food: Practical Applications” (Jan Pokorny, Nedyalka Yanishlieva and Michael Gordon (editors), 2001, Cambridge: CRC Press, Woodhead Publishing Ltd. ISBN 1 85573 463 X, CRC Press, ISBN 0-8493-1221-1). An anti-oxidant is a compound with anti-oxidant activity or a composition with anti-oxidant activity or a composition comprising a compound with anti-oxidant activity, such as activity against the oxidative damage resulting from host immune response. An anti-oxidant for example inhibits oxidation. Oxidation, e.g. reactive oxygen species, in a subject for example induces cellular (oxidative) damage.

The term “health promoting”, such as in ‘health promoting food’, ‘health promoting activity’, ‘health promoting property’, and ‘health promoting potential’, has its regular scientific meaning and here refers to the effect of a compound or a composition, such as the hydrolysed puree or the puree of the invention or obtainable with the method of the invention, on the health of an animal such as a mammal such as a pet animal or a human subject, when such compound or composition is consumed by the animal. Consumption of the compound or composition with health promoting potential contributes to or supports or promotes or increases or maintains the health status of the animal such as a mammal such as a pet animal or a human subject. Reference is made to for example book chapters 1, 5 and 6 of “Antioxidants in Food: Practical Applications” (Jan Pokorny, Nedyalka Yanishlieva and Michael Gordon (editors), 2001, Cambridge: CRC Press, Woodhead Publishing Ltd. ISBN 1 85573 463 X, CRC Press, ISBN 0-8493-1221-1).

With the term “drying” or “dried” in the context of the invention, it is meant that the product obtained upon the drying and the dried product have a moisture content that is 20% or less based on the total weight of the product obtained upon the drying or the dried product, preferably 15% or less, more preferably 10% or less, most preferably 5% or less, such as 0.5%-20%, 1%-18%, 2%-16%, 3%-14%, 4%-12% or 6%-8%. Typically, the product that is dried, e.g. the insect pulp, the aqueous protein fraction, the combination of the solid containing fraction and the aqueous protein fraction, has a moisture content before drying of at least 20% based on the total weight of the product before drying, such as at least 25%, at least 30%, at least 40% or at least 45%.

The present invention will be described with respect to particular embodiments and with reference to certain drawings but the invention is not limited thereto but only by the claims. Furthermore, the terms first, second, third and the like in the description and in the claims, are used for distinguishing between similar elements and not necessarily for describing a sequential or chronological order. The terms are interchangeable under appropriate circumstances and the embodiments of the invention can operate in other sequences than described or illustrated herein.

The embodiments of the invention described herein can operate in combination and cooperation, unless specified otherwise. Moreover, the features of the invention described herein, e.g. features outlined in the context of certain aspects and embodiments, can operate in combination and cooperation, unless specified otherwise.

Furthermore, the various embodiments and features of embodiments, although referred to as “preferred” or “e.g.” or “for example” or “in particular” are to be construed as exemplary manners in which the invention may be implemented rather than as limiting the scope of the invention.

The term “comprising”, used in the claims, should not be interpreted as being restricted to the compounds or steps listed thereafter; it does not exclude other compounds or steps. It needs to be interpreted as specifying the presence of the stated compounds, features, integers, steps or components as referred to, but does not preclude the presence or addition of one or more other compounds, features, integers, steps or components, or groups thereof. Thus, the scope of the expression “a method comprising step A and step B” should not be limited to methods consisting only of steps A and B, rather with respect to the present invention, the only enumerated steps of the method are A and B, and further the claim should be interpreted as including equivalents of those steps. Thus, the scope of the expression “a composition comprising compound A” should not be limited to compositions consisting only of compound A, rather with respect to the present invention, the only enumerated compound comprised by the composition is compound A, and further the claim should be interpreted as including equivalents of that compound.

### BRIEF DESCRIPTION OF THE DRAWINGS

FIG. 1. Glucosamine content of P (pasteurized minced meat (puree) of BSF larvae), HP (hydrolyzed and pasteurized minced meat of BSF), APH (hydrolysate of water-soluble BSF proteins), CM (chicken meal). Results expressed as mean±standard deviation (n=3). Letter ‘a’ above the bars represent significant differences (p<0.05).

FIG. 2. Percentage inhibition of trypsin during hydrolysis of azocasein by P (water-soluble extract of pasteurized minced meat of BSF), HP (water-soluble extract of hydrolyzed and pasteurized minced meat of BSF), APH (hydrolysate of water-soluble BSF proteins), CM (water-soluble extract of chicken meal). Results expressed as mean±standard deviation (n=3).

FIG. 3. Percentage cellular toxicity of P (water-soluble extract of pasteurized minced meat of BSF), HP (water-soluble extract of hydrolyzed and pasteurized minced meat of BSF), APH (hydrolysate of water-soluble BSF proteins), CM (water-soluble extract of chicken meal). Results expressed as mean±standard deviation (n=3). Letter a-d above the bars represent significant differences (p<0.05).

FIG. 4. Reduction in ROS production from macrophages by P (water-soluble extract of pasteurized minced meat of BSF), HP (water-soluble extract of hydrolyzed and pasteurized minced meat of BSF), APH (hydrolysate of water-soluble BSF proteins), CM (water-soluble extract of chicken meal). Results expressed as mean±standard deviation (n=3). Letter a-c above the bars represent significant differences (p<0.05).

FIG. 5. Reduction in ROS production from PMA activated HL-60 cells by P (water-soluble extract of pasteurized minced meat of BSF), HP (water-soluble extract of hydrolyzed and pasteurized minced meat of BSF), APH (hydrolysate of water-soluble BSF proteins), CM (water-soluble extract of chicken meal). Results expressed as mean±standard deviation (n=3). Letter a or b above the bars represent significant differences (p<0.05).

FIG. 6. Cellular toxicity of P (water-soluble extract of pasteurized minced meat of BSF), HP (water-soluble extract of hydrolyzed and pasteurized minced meat of BSF), APH (hydrolysate of water-soluble BSF proteins), CM (water-soluble extract of chicken meal). Results expressed as mean±standard deviation (n=3).

FIG. 7: DPPH radical scavenging activity of BSF PureeX™ (BSF-P), BSF hydrolyzed puree (BSF-HP), Chicken meal (CM) after 30 min incubation (n=3). FM: fish meal control.

FIG. 8: DPPH radical scavenging activity of BSF PureeX™ (BSF-P), BSF hydrolyzed puree (BSF-HP), Chicken meal (CM) after 60 min incubation (n=3). FM: fish meal control.

FIG. 9: ABTS cation radical scavenging activity of BSF PureeX™ (BSF-P), BSF hydrolyzed puree (BSF-HP), Chicken meal (CM) after 30 min incubation (n=3). FM: fish meal control.

FIG. 10: MPO response modulation activity of BSF PureeX™ (BSF-P), BSF hydrolyzed puree (BSF-HP), Chicken meal (CM) using SIEFED assay (n=3). FM: fish meal control.

FIG. 11 MPO response modulation activity of BSF PureeX™ (BSF-P), BSF hydrolyzed puree (BSF-HP), Chicken meal (CM) using classical measurement (n=3). FM: fish meal control.

FIG. 12: Neutrophil response modulation activity of BSF PureeX™ (BSF-P), BSF hydrolyzed puree (BSF-HP), Chicken meal (CM) (n=3). FM: fish meal control.

FIG. 13: DPPH radical scavenging activity of BSF aqueous protein hydrolysate (BSF-APH), Chicken meal (CM) and Fish meal (FM) after 30 min incubation (n=3).

FIG. 14: DPPH radical scavenging activity of BSF aqueous protein hydrolysate (BSF-APH), Chicken meal (CM) and Fish meal (FM) after 60 min incubation (n=3).

FIG. 15: ABTS cation radical scavenging activity of BSF aqueous protein hydrolysate (BSF-APH), Chicken meal (CM) and Fish meal (FM) after 30 min incubation (n=3).

FIG. 16: MPO response modulation activity of BSF aqueous protein hydrolysate (BSF-APH), Chicken meal (CM) and Fish meal (FM) using SIEFED assay (n=3).

FIG. 17: MPO response modulation activity of BSF aqueous protein hydrolysate (BSF-APH), Chicken meal (CM) and Fish meal (FM) using classical measurement (n=3).

FIG. 18: Neutrophil response modulation activity of BSF aqueous protein hydrolysate (BSF-APH), Chicken meal (CM) and Fish meal (FM) (n=3).

### DETAILED DESCRIPTION

It is a first goal of the present invention to provide an improved compound or composition or food or feed ingredient or product or food or feed supplement or pharmaceutical composition, for administration to a mammal at risk for developing osteoarthritis or suffering from osteoarthritis, such as a suitable bioactive substance or composition.

It is an objective of the current invention to provide an improved compound or composition or food or feed ingredient or product or food or feed supplement or pharmaceutical composition, for convenient intake, preferably suitable for oral administration, and/or which addresses, reverses, inhibits, silences, etc., at least one and preferably more than one of the factors involved, causing and contributing to the multifactorial disease, i.e. osteoarthritis.

At least one of the above objectives is achieved by providing a protein composition isolated from processed black soldier fly larvae, of the invention.

The present invention will be described with respect to particular embodiments but the invention is not limited thereto but only by the claims. The embodiments of the invention described herein can operate in combination and cooperation, unless specified otherwise.

Osteoarthritis (OA) is a multifactorial disease. Progression of OA is related to oxidative stress and reactive oxygen species (ROS). Augmented ROS formation is at the basis of damaging structural biomolecules of a joint inflicted by OA. ROS are involved in inflammatory responses and as such contribute to joint inflammation, synovitis, and as a consequence, pain and restricted movement of the joint. The OA is a chronic joint disease, accompanied by amongst others cartilage degradation, synovial inflammation, subchondral bone remodeling and osteophyte formation. Oxidative stress and ROS are pro-inflammatory mediators, contributing to the occurring and progression of OA. The OA is characterized by morphological, biochemical, molecular and biomechanical changes of both cells and extracellular matrix (ECM) which leads to softness, loss of articular cartilage, synovial inflammation, sclerosis of subchondral bone, formation of osteophytes and subchondral cysts. Several of the many hallmarks of OA are bloody synovial fluid, e.g. accompanied by sedimented and/or lysed erythrocytes, increased number of neutrophils in the synovial fluid of the affected joint, increased white blood-cell count in the synovial fluid, all compared to the synovial fluid of a joint of a healthy subject (human, animal, pet, such as a human subject or a horse, dog, cat) not suffering from arthritis, notably OA.

Although the cause of OA is still not completely understood, a combination of mechanical stress on a joint and low-grade inflammation are thought to be at the basis of the onset and progression of OA. As said, multiple aspects and processes contribute to the development and progression of the OA, of which presence of oxidative stress, build-up of ROS in the synovium of the joint, triggering and activation of the innate immune system, activation of synovial macrophages, red blood-cell accumulation and/or lysis and/or sedimentation in the synovial fluid of the joint, joint-space narrowing, etc., are some important contributors to the disease phenotype: characterized by amongst others, pain, joint stiffness, swelling of the joint, all for example as a consequence of inflammation and deterioration of articular cartilage and even in severe cases the underlying bone.

Single-molecule, single-target OA therapies are under development. For example, ROS targeting with small molecules is a current approach under investigation for knee OA. For example, orally administered small molecules are tested as a therapy for relieving the inflammation component of the disabilitating and progressive disease, OA. Lowering ROS formation is at the heart of this approach. However, since OA is a multifactorial disease, targeting a single pathway or a single molecule is most likely not sufficient and enough for combating OA and for providing a sufficiently high degree of relief from e.g. pain, stiffness, swelling, etc. such that the quality of life of the subject suffering from joint inflammation, synovitis, cartilage damage in the joint, an arthritis, OA, etc., improves to a desired level, although of course some improvement could perhaps be expected from single compound/single (bio)molecular target molecule or pathway.

The inventors now came to the surprising insight that a protein composition derived from black soldier fly larvae exerts multiple biological activities on cells and biological pathways relating to joint biology, which in combination provides for a highly effective and beneficial treatment option for combating OA onset, OA and OA progression, when administered to a subject such as a human or an animal, in a prophylactic setting or when such a subject is an OA patient suffering from OA in at least one joint (e.g. ankle, back, shoulder, knee, wrist, hip, thumb, etc., that is to say, suffering from (osteo)arthritis affecting at least one synovial joint, i.e. one or more of the most moveable joints of a vertebrate). According to the invention, such administering of the protein composition is preferably the oral administration to the subject, e.g. as a food ingredient, a feed ingredient. Thus, the inventors established that administering the (hydrolysed) insect protein composition (e.g. as a food or feed ingredient, e.g. as part of a foodstuff, or food or feed supplement) to a subject results in systemic effects, that is to say, the oral administration relates to inducing the systemic effects and exerting multiple systemic biological activities on cells and biological pathways relating to joint biology. Oral administration thus provides for a highly effective and beneficial systemic treatment option for combating OA onset, OA and OA progression, when orally administered to a subject such as a human or an animal, in a prophylactic setting or when such a subject is an OA patient suffering from OA in at least one joint (e.g. ankle, back, shoulder, knee, wrist, hip, thumb, etc., that is to say, suffering from (osteo) arthritis affecting at least one synovial joint, i.e. one or more of the most moveable joints of a vertebrate). The inventors identified several protein fractions and extracts isolated from BSF larvae, which display an inhibitory activity towards cellular ROS formation. In addition, such protein compositions exerts (synovium) macrophage inhibitory activity, resulting in preventing macrophages from becoming activated and/or in silencing activated macrophages, which both result in preventing or dampening or halting or inhibiting a pro-inflammatory response and/or an immune response e.g. in the joint synovium. Moreover, the inventors established that the protein compositions such as (hydrolysed) BSF larvae protein and water soluble and/or hydrolysed BSF larvae protein, contain an amount of glucosamine beneficial to pain relief and reversal or prevention of joint-space narrowing, when such protein composition is administered to a patient (human, pet, horse, dog, etc.) suffering from joint inflammation, OA, progressed OA. In addition, the inventors also established that such protein compositions derived from BSF larvae puree (minced and heated larvae, e.g. 10-18 days post hatching, e.g. 1-3 days before pre-pupation) have a protective activity when susceptibility of cells to lyse is considered. That is to say, cell are prevented from cell lysis when contacted with the proteins or hydrolysed proteins or water-soluble fraction of BSF proteins or hydrolysed water-soluble fraction of BSF proteins or water-soluble fraction of hydrolysed BSF proteins. Herewith, the cells remain undamaged and do not contribute to a pro-inflammatory response in e.g. a joint at risk of becoming inflicted with OA or in an inflamed joint or a joint with OA. In combination, the anti-ROS forming activity, the prevention of macrophage activation, the prevention or treatment or inhibition of damage to a joint induced by macrophage activation, the prevention of cell lysis and the presence of glucosamine, contribute to a multifactorial treatment modality or prophylaxis modality when the use of the BSF insect protein composition for the treatment or prevention of joint inflammation, innate immune response activity in the joint, onset of OA, progression of OA, synovitis, pain, swelling, stiffness, low-grade inflammation, etc., is considered. That is to say, the insect protein composition provides for the first time for a treatment option that addresses more than one of the several factors contributing to disease onset or progression and/or disease symptoms. By (orally) administering such a protein composition, a mammal is not only prevented from developing or treated for e.g. low-grade inflammation in a joint or OA, as a consequence of innate-immune system activation and/or macrophage activation, but at the same time, also (red blood-cell) lysis in the synovial fluid is prevented and/or (excessive) ROS formation is halted or prevented, and/or the provision of glucosamine or glucosamine-sulphate may contribute to pain prevention or relief and/or reversing or preventing joint-space narrowing. This way, by aiming at silencing, preventing, inhibiting, reversing, halting, etc., multiple molecules, cells, pathways, contributing to disease, e.g. OA, onset and progression, the healthy subject such as a human subject, animal such as a horse or a pet such as a dog or a cat, is improvingly prevented from the occurrence of OA. In addition and similarly, by aiming at silencing, preventing, inhibiting, reversing, halting, etc., multiple molecules, cells, pathways, contributing to disease progression, e.g. OA joint inflammation, etc., progression, the patient suffering from such dis-abilitating disease such as a human OA patient, animal such as a horse or a pet such as a dog or a cat suffering from OA, is improvingly treated from the e.g. OA and/or at least the disease symptoms such as cartilage-damage related pain, joint pain in general, impaired joint movement, joint swelling or stiffness, etc.

An aspect of the invention relates to an insect protein composition for use as a medicament. The medicament is preferably for oral use. The medicament is preferably orally administered to the subject in need of such use of the insect protein composition. To the knowledge of the inventors, this is the first time that an insect protein composition such as a protein composition derived from minced BSF larvae such as a water-soluble fraction and/or enzymatically hydrolysed fraction, is applied for conquering at least one and preferably several aspects of a disease, here amongst others joint inflammation and OA onset and progression. The insect protein composition, such as BSF protein composition, e.g. derived from minced and pasteurized (heated) BSF larvae, is suitable for preventing and/or reversing or inhibiting causes and consequences of e.g. OA, e.g. when orally administered, such as administered as part of a diet. That is to say, administering the protein composition to a healthy subject at risk for developing OA or to a subject suffering from OA, e.g. a human subject, an animal such as a pet or a horse, preferably a dog, inhibits pathways such as cellular pathways contributing to ROS formation, innate immune system activation, cell lysis, macrophage activation, therewith treating or preventing joint inflammation, pain, swelling, stiffness, etc. For any of the here-aforementioned used and the applications and uses outlined hereafter, such use implies preferably the oral administration of the insect protein composition, wherein for example the protein is enzymatically hydrolysed before such oral administration. For example, a foodstuff comprises (hydrolysed) insect protein composition, and/or the (hydrolysed) insect protein composition is an ingredient of a feed for an animal, or a food for a human subject. The use of the (hydrolysed) protein composition is for example the oral administration of the (hydrolysed) protein composition (e.g. as an ingredient of a foodstuff) as part of a (daily) diet of a subject such as a human subject or an animal such as a pet such as a cat or a dog.

An aspect of the invention relates to an insect protein composition for use in a method for the prophylaxis or treatment of inflammation.

An aspect of the invention relates to an insect protein composition for use in a method for the prophylaxis or treatment of inflammation in a joint, such as in a joint affected by osteoarthritis.

An aspect of the invention relates to an insect protein composition for use in a method for the prophylaxis or treatment of synovial inflammation and/or synovitis, such as in a joint affected by osteoarthritis.

An aspect of the invention relates to an insect protein composition for use in a method for the prophylaxis or treatment of damage of structural biomolecules of a joint, such as in a joint affected by osteoarthritis.

An aspect of the invention relates to an insect protein composition for use in a method for the prophylaxis or treatment of chondrocyte apoptosis, such as in a joint affected by osteoarthritis.

An aspect of the invention relates to an insect protein composition for use in a method for the prophylaxis or treatment of dysfunction of a subchondral bone and/or of subchondral bone remodeling and/or of bone loss, such as of a bone of a joint affected by osteoarthritis.

An aspect of the invention relates to an insect protein composition for use in a method for the prophylaxis or treatment of cartilage degradation, such as of cartilage of a joint affected by osteoarthritis. An aspect of the invention relates to an insect protein composition for use in a method for the prophylaxis or treatment of osteophyte formation, such as in a joint affected by osteoarthritis.

An aspect of the invention relates to an insect protein composition for use in a method for the prophylaxis or treatment of loss of articular cartilage, such as in a joint affected by osteoarthritis.

An aspect of the invention relates to an insect protein composition for use in a method for the prophylaxis or treatment of sclerosis of subchondral bone, such as a bone of a joint affected by osteoarthritis.

An aspect of the invention relates to an insect protein composition for use in a method for the prophylaxis or treatment of subchondral cysts formation, such as in a joint affected by osteoarthritis.

An aspect of the invention relates to an insect protein composition for use in a method for the prophylaxis or treatment of synovial macrophage activation and/or (activated) synovial macrophage-induced damage, such as in synovium of a joint affected by osteoarthritis.

An aspect of the invention relates to an insect protein composition for use in a method for the prevention or treatment of macrophage-induced synovial damage, such as in synovium of a joint affected by osteoarthritis.

An aspect of the invention relates to an insect protein composition for use in a method for the prophylaxis or treatment of low-grade inflammatory disease of a joint, such as of a joint affected by osteoarthritis.

An aspect of the invention relates to an insect protein composition for use in a method for the prophylaxis or treatment of joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis.

An aspect of the invention relates to an insect protein composition for use in a method for the prophylaxis or treatment of innate immune response in a joint, such as in a joint affected by osteoarthritis.

An aspect of the invention relates to an insect protein composition for use in a method for the prophylaxis or treatment of joint stiffness and/or locomotor restriction and/or flexion contracture and/or decreased flexion, such as of a joint affected by osteoarthritis.

An aspect of the invention relates to an insect protein composition for use in a method for the prophylaxis or treatment of synovial fibrosis, such as in a joint affected by osteoarthritis.

An aspect of the invention relates to an insect protein composition for use in a method for the prophylaxis or treatment of joint-space narrowing, such as in a joint affected by osteoarthritis.

An aspect of the invention relates to an insect protein composition for use in a method for the prophylaxis or treatment of deterioration of articular cartilage and underlying bone, such as in a joint affected by osteoarthritis.

An aspect of the invention relates to an insect protein composition for use in a method for the prophylaxis or treatment of erythrocyte lysis and/or elevated erythrocyte sedimentation compared to normal, such as erythrocyte lysis and/or elevated erythrocyte sedimentation in synovium of a joint affected by osteoarthritis.

An aspect of the invention relates to an insect protein composition for use in a method for the prophylaxis or treatment of arthritis.

An aspect of the invention relates to an insect protein composition for use in a method for the prophylaxis or treatment of osteoarthritis.

An aspect of the invention relates to an insect protein composition for use in a method for the prophylaxis or treatment of osteoarthritis progression.

An aspect of the invention relates to an insect protein composition for use in a method for the prophylaxis or treatment of erosive osteoarthritis or inflammatory osteoarthritis.

An aspect of the invention relates to an insect protein composition for use in a method for the prophylaxis or treatment of early-stage osteoarthritis or late-stage osteoarthritis.

An embodiment according to the invention is the insect protein composition for use in a method for the prophylaxis or treatment of:
inflammation; and/or
inflammation in a joint, such as in a joint affected by osteoarthritis; and/or
synovial inflammation and/or synovitis, such as in a joint affected by osteoarthritis; and/or
damage of structural biomolecules of a joint, such as in a joint affected by osteoarthritis;
and/or
chondrocyte apoptosis, such as in a joint affected by osteoarthritis; and/or
dysfunction of a subchondral bone and/or of subchondral bone remodeling and/or of bone loss, such as of a bone of a joint affected by osteoarthritis; and/or
cartilage degradation, such as of cartilage of a joint affected by osteoarthritis; and/or osteophyte formation, such as in a joint affected by osteoarthritis; and/or loss of articular cartilage, such as in a joint affected by osteoarthritis; and/or
sclerosis of subchondral bone, such as a bone of a joint affected by osteoarthritis; and/or
subchondral cysts formation, such as in a joint affected by osteoarthritis; and/or
synovial macrophage activation and/or (activated) synovial macrophage-induced damage, such as in synovium of a joint affected by osteoarthritis; and/or
macrophage-induced synovial damage, such as in synovium of a joint affected by osteoarthritis; and/or
low-grade inflammatory disease of a joint, such as of a joint affected by osteoarthritis; and/or
joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis; and/or
innate immune response in a joint, such as in a joint affected by osteoarthritis; and/or joint stiffness and/or locomotor restriction and/or flexion contracture and/or decreased flexion, such as of a joint affected by osteoarthritis; and/or
synovial fibrosis, such as in a joint affected by osteoarthritis; and/or
joint-space narrowing, such as in a joint affected by osteoarthritis; and/or
deterioration of articular cartilage and underlying bone, such as in a joint affected by osteoarthritis; and/or
erythrocyte lysis and/or elevated erythrocyte sedimentation compared to normal, such as erythrocyte lysis and/or elevated erythrocyte sedimentation in synovium of a joint affected by osteoarthritis; and/or
arthritis; and/or
osteoarthritis; and/or
osteoarthritis progression; and/or
erosive osteoarthritis or inflammatory osteoarthritis; and/or
early-stage osteoarthritis or late-stage osteoarthritis.

Preferred is the insect protein composition for use according to the invention, wherein the insect is larvae of black soldier fly. Such larvae are for example cultured for 8-20 days post-hatching and preferably processed for obtaining the protein composition at a time-point 1-3 days before such larvae transform into prepupae and pupae. The inventors established that BSF larvae are particularly suitable as a source of proteins, protein extracts or fractions, water-soluble proteins, hydrolysed proteins, preferably enzymatically hydrolysed proteins, water-soluble hydrolysed proteins, enzymatically hydrolysed water-soluble proteins, endowed with the capacity to silence or inhibit e.g. ROS formation by cells, macrophage activation, (erythrocyte) lysis, (joint) inflammation. In addition, protein compositions obtained from insect species other than BSF larvae may have the same or similar bioactivity related to prophylaxis or treatment of any of the here-above listed disease symptoms, OA, synovial inflammation, etc., when administered to a subject such as a human or dog.

Preferred is the insect protein composition for use according to any one of the aforementioned embodiments relating to disease symptoms and disease, e.g. OA, wherein the insect is larvae of black soldier fly. Also preferred is the insect protein composition for use according to the invention, wherein the insect protein composition is minced and heated insects, preferably black soldier fly, more preferably larvae of black soldier fly. According to embodiments of the invention the insect protein composition for use according to the invention is enzymatically hydrolyzed minced and heated insects, preferably black soldier fly, more preferably larvae of black soldier fly. In particular, the insect protein composition for use according to the invention is the water-soluble extract of minced and heated insects, preferably black soldier fly, more preferably larvae of black soldier fly, or the water-soluble extract of enzymatically hydrolyzed minced and heated insects, preferably black soldier fly, more preferably larvae of black soldier fly. Also preferred is the insect protein composition for use according to the invention, wherein the insect protein composition is the enzymatically hydrolyzed water-soluble extract of minced and heated insects, preferably black soldier fly, more preferably larvae of black soldier fly. An embodiment is the insect protein composition for use according to the invention, wherein the insect protein composition is heated enzymatically hydrolyzed minced insects, preferably black soldier fly, more preferably larvae of black soldier fly. An embodiment is the insect protein composition for use according to the invention, wherein the insect protein composition is the water-soluble extract of heated enzymatically hydrolyzed minced insects, preferably black soldier fly, more preferably larvae of black soldier fly.

Preferred is the insect protein composition for use according to the invention, wherein the insect protein composition is orally administered.

Another aspect of the present invention features a pharmaceutical composition comprising a BSF insect protein composition according to the invention and a physiologically acceptable carrier. A “pharmacological composition” refers to a composition in a form suitable for administration into a mammal, preferably a human, a horse, a pet such as a dog or a cat, preferably a human subject or a dog. Preferably, the pharmaceutical composition contains a sufficient amount of the insect protein composition according to the invention in a proper pharmaceutical form to exert a therapeutic effect on a human or on an animal such as a horse, pet, such as a dog or a cat, preferably a dog.

Considerations concerning forms suitable for administration are known in the art and include toxic effects, solubility, route of administration, and maintaining activity. For example, pharmacological compositions injected into the blood stream should be soluble. However, preferred is the oral route of administration. For example, the pharmaceutical composition is provided as a powder, tablet, capsule.

Suitable dosage forms, in part depend upon the use or the route of entry, for example oral, transdermal or by injection. Such dosage forms should allow a pharmaceutically active compound to reach a target cell whether the target cell is present in a multicellular host or in a culture. Factors are known in the art, and include considerations such as toxicity and dosage form which retard the compound or composition from exerting its effect.

While the invention has been described in terms of several embodiments, it is contemplated that alternatives, modifications, permutations and equivalents thereof will become apparent to one having ordinary skill in the art upon reading the specification and upon study of the drawings. The invention is not limited in any way to the illustrated embodiments. Changes can be made without departing from the scope which is defined by the appended claims.

An embodiment is the insect protein composition for use according to the invention, wherein the insect protein composition is administered to a mammal.

Particularly preferred is the insect protein composition for use according to the invention, wherein the insect protein composition is administered to a human subject or to a human patient, such as a human patient suffering from osteoarthritis. Also particularly preferred is the insect protein composition for use according to the invention, wherein the insect protein composition is administered to a dog, such as a dog suffering from osteoarthritis. An embodiment is the insect protein composition for use according to the invention, wherein the insect protein composition is administered to an animal, preferably a mammalian animal, e.g. a horse, a pet.

Preferred is the insect protein composition for use according to the invention, wherein the insect protein composition is administered to a horse, a pet such as a dog or a cat, such as a horse, a pet such as a dog or a cat suffering from osteoarthritis.

In particular, the insect protein composition for use according to the invention is (orally) administered to a human patient suffering from any one or more of: inflammation, for example inflammation in a joint, such as in a joint affected by osteoarthritis, synovial inflammation and/or synovitis, such as in a joint affected by osteoarthritis, damage of structural biomolecules of a joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation, activated macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis. Also preferred is the insect protein composition for use according to the invention, wherein the insect protein composition, preferably derived from BSF larvae, is (orally) administered to a dog suffering from any one or more of these diseases and/or disease symptoms, such as synovial inflammation, low-grade inflammation, joint pain, OA, in particular OA.

As said, the inventors surprisingly established that the BSF larvae-derived protein composition (e.g. hydrolysed protein, water-soluble protein extract, enzymatically hydrolysed water-soluble protein extract, or water-soluble enzymatically hydrolysed BSF larvae protein) has one or several activities relating to preventing and/or treating low-grade joint inflammation, OA, joint pain, or limited joint movement, relating to one or more of (a) inhibiting macrophage activation and/or preventing macrophage activation and/or inhibiting and/or preventing (activated) macrophage-induced damage to e.g. synovium, cartilage, the underlying bone; (b) preventing and/or inhibiting cell lysis such as erythrocyte lysis; and (c) inhibiting and/or preventing reactive oxygen species formation by a cell such as an endothelial cell or a macrophage, and/or comprises glucosamine and/or glucosamine-sulphate suitable for relief of OA related pain and/or suitable for preventing or reversing joint-space narrowing. Therefore, preferred is the insect protein composition for use according to the invention, wherein the insect protein composition has any one or more of the following activities: (a) inhibiting macrophage activation and/or preventing macrophage activation, and/or inhibiting and/or preventing (activated) macrophage-induced damage to e.g. synovium, cartilage, the underlying bone, in particular preventing or inhibiting macrophage-induced synovial damage; (b) preventing and/or inhibiting cell lysis such as erythrocyte lysis; and (c) inhibiting and/or preventing reactive oxygen species formation by a cell such as an endothelial cell or a macrophage, and/or comprises glucosamine and/or glucosamine-sulphate. Thus, an aspect of the invention relates to an insect protein composition for use in a method for the prevention or treatment of macrophage-induced synovial damage, such as in synovium of a joint affected by osteoarthritis. The amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

Particularly preferred is the insect protein composition for use according to the invention, wherein the insect protein composition inhibits and/or prevents macrophage activation and/or prevents or inhibits macrophage-induced synovial damage; prevents and/or inhibits reactive oxygen species formation by a cell such as an endothelial cell or a macrophage; and comprises glucosamine. Preferably, the insect protein composition is obtained from minced and pasteurized BSF larvae. The insect protein composition can be the water-soluble fraction of such processed larvae, and/or can be the enzymatically hydrolysed product of such processed larvae of BSF. The amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

An aspect of the invention relates to a human food supplement or animal feed supplement, human food ingredient or animal feed ingredient or human food product or animal feed product, comprising the insect protein composition of the invention. Optionally, said insect protein composition, e.g. protein composition derived from black soldier fly larvae, comprises glucosamine. If present in the insect protein composition, the amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

An aspect of the invention relates to use of the insect protein composition according to the invention in the preparation of a human food supplement or animal feed supplement, human food ingredient or animal feed ingredient or human food product or animal feed product. Optionally, said insect protein composition, e.g. protein composition derived from black soldier fly larvae, comprises glucosamine. If present in the insect protein composition, the amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

Preferred is the animal feed supplement, ingredient or product of the invention or use of the insect protein composition in the preparation of an animal feed supplement, ingredient or product according to the invention, wherein the animal is a horse or a pet such as a dog or a cat. Preferably, the mammal is a dog.

An aspect of the invention relates to a non-therapeutic method of prevention against, providing relief from or amelioration of a joint problem in a human subject or an animal such as a horse, a pet such as a dog or a cat, preferably a human subject or a dog, the method comprising orally administering to the human subject or the animal the insect protein composition of the invention or the human food supplement or animal feed supplement, human food ingredient or animal feed ingredient or human food product or animal feed product of the invention, wherein the joint problem is any one or more of: inflammation, for example inflammation in a joint affected by osteoarthritis, synovial inflammation and/or synovitis, such as in a joint affected by osteoarthritis, damage of structural biomolecules of the joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation and/or macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis. The human subject is a healthy human subject not suffering from OA, or is a patient with OA; the animal, for example a horse or a dog, preferably a dog, does not suffer from OA, or suffers from OA. Optionally, said insect protein composition applied in the non-therapeutic method, e.g. protein composition derived from black soldier fly larvae, comprises glucosamine. The amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

Particularly suitable is the non-therapeutic method of prevention against, providing relief from or amelioration of a joint problem in a human subject or an animal such as a horse, a pet such as a dog or a cat, preferably a human subject or a dog according to the invention, wherein the joint problem is osteoarthritis and wherein the human subject is a human subject not suffering from osteoarthritis or is a human patient suffering from osteoarthritis, and wherein the animal such as a horse, dog or cat, preferably a dog, is a dog not suffering from osteoarthritis or is a dog suffering from osteoarthritis.

An aspect of the invention relates to a pharmaceutical composition comprising the insect protein composition of the invention and optionally a pharmaceutically acceptable diluent and/or a pharmaceutically acceptable excipient. Preferably, the pharmaceutical composition is formulated for oral administration. Suitable oral formulations are a powder, a tablet, a capsule. Optionally, said insect protein composition comprised by the pharmaceutical composition, e.g. protein composition derived from black soldier fly larvae, comprises glucosamine. The amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

An aspect of the invention relates to the pharmaceutical composition of the invention for use according to the invention. That is to say, an aspect of the invention relates to a pharmaceutical composition (preferably for oral administration) comprising the insect protein composition of the invention, such as a protein composition derived from BSF larvae, for use in a method for the treatment or prophylaxis of any one or more of: inflammation, for example inflammation in a joint affected by osteoarthritis, synovial inflammation and/or synovitis, such as in a joint affected by osteoarthritis, damage of structural biomolecules of the joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation and/or macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis. For example, the pharmaceutical composition is administered to a human subject or to a mammalian animal. The human subject is a healthy human subject not suffering from OA, or is a patient with OA; the animal, for example a horse or a dog, preferably a dog, does not suffer from OA, or suffers from OA. Optionally, said insect protein composition comprised by the pharmaceutical composition, e.g. protein composition derived from black soldier fly larvae, comprises glucosamine. The amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

Preferred is the pharmaceutical composition for use in a method for the treatment of an arthritis, preferably OA. Also preferred is the pharmaceutical composition for use in a method of treatment of joint inflammation. Furthermore, the pharmaceutical composition is for example for use in a method to inhibit macrophage activation e.g. in the synovium of a subject such as a human patient suffering from OA or a pet such as a dog, to silence innate immune response, in particular in an inflamed joint such as a joint of an OA patient (human, animal, e.g. pet, horse, cat, dog).

Preferred is the pharmaceutical composition for use of the invention in a method of treatment or prophylaxis of any one or more of osteoarthritis, osteoarthritis progression, erosive osteoarthritis, inflammatory osteoarthritis, early-stage osteoarthritis or late-stage osteoarthritis, wherein a human subject is treated or an animal such as a horse or a pet, for example a dog or a cat, preferably a human patient or a dog, such as a human patient or a dog suffering from osteoarthritis.

Black soldier fly larvae (BSF; Hermetia illucens) derived proteins are gaining popularity as sustainable pet food and animal feed ingredients. These ingredients are nutritious, highly digestible and promote health of consuming mammals such as animals. Currently, pet food is the biggest market for insect proteins in Europe. Globally, 50% of households own a cat or dog. These two companion animals are together responsible for 95% of the global pet food sales. Health and wellbeing of these companion animals are of prime importance to their owners.

The inventors now established the preventive activity of BSF protein compositions and BSF protein derivatives (hydrolysate of BSF proteins, hydrolysate of water-soluble BSF proteins, water soluble extract of hydrolysed BSF protein), isolates and extracts in various pathways involved in developing arthritis and pathways leading to arthritis formation. In vitro assays were applied to establish the treating and prophylaxis of arthritis with BSF protein, derivatives, hydrolysates, compositions, isolates and extracts. To the best of the knowledge of the inventors, this is for the first time that the anti-arthritis potential of insect proteins such as BSF proteins and extracts and hydrolysates thereof has been established. Chick meal is commonly used in pet food formulations as a protein source and hence was used as an industrial benchmark in the examples with BSF proteins and extracts and hydrolysates thereof. Surprisingly, BSF protein compositions and hydrolysates and water-soluble extracts thereof are suitable for use in a method for the prophylaxis and/or treatment of inflammation. Typically, the protein is derived from BSF larvae, such as minced and pasteurized larvae. According to the invention, a suitable source of protein for use in a method for preventing or treating inflammation or symptoms and health problems occurring as a consequence of inflammation or arthritis, e.g. OA, is protein derived from BSF larvae that are 5-25 days of age post hatching and/or that are 1-3 days before pre-pupation phase. Moreover, and more specifically, the inventors demonstrate that such proteins isolated from BSF larvae and hydrolysates and water-soluble isolates or extracts thereof and water-soluble protein extracts thereof that are hydrolysed, are suitable for use in a method for treating and/or preventing arthritis, such as OA. Moreover, such compositions are effective in relief from symptoms such as inflammation and pain, accompanying arthritis such as OA, when used in a method of treatment of arthritis, OA, inflammation, pain related to arthritis or OA.

Detrimental reduction of the wellbeing of companion animals due to diseases may have a negative impact on the owner's pet-companion relationship. In veterinary sciences, osteoarthritis (OA) represents the most commonly diagnosed joint disease. It is characterized by chronic, painful, degenerative and inflammatory disease affecting synovial diarthrosis joints, which are freely movable point of contact between two bones (e.g. hips or knee). Based on data from North America, it was suggested that up to 50% of dogs from 8 to 13 years old are affected by OA. Being 20% of dogs above one year affected by OA and 80% dogs affected when older than 8 year. Despair the distress of the diseases affecting both pet and consequently pet's owner on daily pet's activities (e.g. dog's pain while playing, walking or changes in faecal and urinary habits), there is also the economic burden associated to the treatment of the diseases. In humans, the average osteoarthritic-related cost is $9,882 per person in a year while in horses the cost was on average $10,000. Similarly, human arthritis patients such as OA patients suffer from pain, inflammation, disability to move joints freely and unencumbered, etc. An effective cure or pain-relieving or protective therapy, preferably with little or without side effects, is currently lacking for mammals such as pets and human subjects, such as dogs, cats, etc.

The cause of OA in human patients and pets such as dogs is largely unknown, although aging, obesity and traumas or luxations have been pinpointed as likely and the most common risk factors for the development and progression of the disease. Arthritis such as OA affects both humans as well as other animals (e.g. dogs or horses) and the disease is still far from being completely understood. Due to similarities between humans and dogs in the onset and progression of OA, dogs are commonly used as canine model for the study of this disease (Robertson-Plouch et al., 2019). Histologically, OA is characterized by: a) degradation of the articular cartilage, b) thickening of the subchondral bone, c) osteophyte formation and d) synovial inflammation (synovitis) (Loeser et al., 2012). Healthy synovial di-arthrodial joints are comprised of different scaffolds, which are tightly regulated to support the join well-functioning to behave in a dynamic and flexible way and to being able to support strong forces. In this type of joint three key structural components are located: a) Articular capsule, tissue limiting the joint environment; b) Articular cartilage, layer coating the end of the bones at the joint's location and c) Joint cavity which contains the synovial fluid (SF).

The articular capsule contains specialized cells, type a synoviocytes (macrophages-like cells) and type b synoviocytes (fibroblast-like cells). These cells are responsible for processing debris and pathogens (type a synoviocytes), producing enzymes or relevant molecules used as building block of the articular cartilage (e.g. hyaluronic acid) (type b synoviocytes). Both cell types can produce signalling molecules such as cytokines. The alternation in the molecule homeostasis in this tissue can initiate synovitis, characterized by the infiltration of macrophages in places in the joint where they usually would not be present. This is a feature of OA.

Articular cartilage is a tissue lacking nerves, lymphatic system or blood vessels, showing a limited capacity of repair. This tissue is formed by chondrocytes (<1% wet weight of cartilage) which are embedded in the ExtraCellular Matrix (ECM), which they produce and maintain. Three main elements constitute the ECM: a) Collagen (20% wet weight cartilage), b) Proteoglycans (5% wet weight cartilage), and c) Water (70-75% wet weight cartilage). Protein, lipids and phospholipids and other molecules are also present in a minor extent. External factors (e.g. growth factors) can influence chondrocytes metabolism, resulting in chondrocytes production of molecules associated with the synthesis or degradation (proteinases or metalloproteinases) of the ECM. An imbalance in the chondrocyte metabolism is associated to OA.

Additionally, associated to OA, fibrosis may occur. This process is a trigger by inflammation and tissue damage and it results in excessive ECM formation causing joint stiffness and thickness at the early stages of OA.

The synovial fluid (SF) is an ultra-filtrate of plasma containing lubricant molecules (proteoglycan or hyaluronan), secreted by neighbouring synoviocytes (synovial lining cells). This SF has two functions: 1) Transport of medium with nutrients, waste, enzymes and 2) Communication medium, in which soluble molecules such as cytokines or growth factors are present. Changes in the normal composition or metabolism of the SF are noticed in the diseases and pathologies associated with joints such as OA.

At the latest stages of OA and stimulated by the new vascular network infiltrating into the articular cartilage, new bone projections (named osteophytes) are formed. Rather than contributing to the progression of the disease, osteophytes counterbalance the articular degradation and stabilize the joint.

Due to the complexity of OA and accompanying symptoms and health problems (e.g. pain, inflammation, inability to freely and pain-free movement of joints, etc.), experts in the field expect that a single treatment e.g. with a single pharmaceutical compound will not be effective and sufficient in targeting and treating and/or preventing arthritis such as OA and/or its symptoms. The literature available on the different treatment options to treat OA and/or OA symptoms are limited and with not very compelling study design. Most of the current treatments available focus on alleviating the symptoms (pain) rather than treating the disease.

The current treatments available are:

Use of non-Steroidal Anti-Inflammatory Drugs (NSAIDs), for their effectiveness and easy administration. These NSAIDs act by inhibiting the cyclooxygenase (COX), which results in a decrease in pro-inflammatory prostaglandins. However, adverse health effects in the gastro-intestinal tract (GI) may commonly occur.

Nutritional supplements comprising one or more of chondroitin sulphate, glucosamine sulphate and hyaluronic acid, which molecules may have the following main mode of actions: 1) building block (glycosamino-glycans; GAG) of aggrecan and 2) anti-inflammatory and anti-catabolic effect by blocking NF-kB pathway. One of the drawbacks of current nutritional supplements comprising such molecules is the poor bioavailability of such compounds.

Green lipped mussels preparation which may have anti-inflammatory properties.

There is a correlation with obesity and OA development. Omega 3-poly-unsaturated fatty acids (PUFA) have been shown to have a positive effect in treatment of OA. In contrast, for mono-unsaturated fatty acids there are inconclusive findings on the putative beneficial effect on OA.

Avocado-soybean un-saponifiables (ASU), and more in particular, the main components of ASU which are fat-soluble vitamins, phyto-esterols, triterpene alcohols and furan fatty acids may have beneficial effects in OA patients. Based on in vitro studies, for ASU it has been reported that components may inhibit metalloproteases, inhibit TNF-β (tumor necrosis factor B), IL-1 (interleukin-1), COX-2 (cyclooxygenase-2), iNOS (inducible Nitric Oxide Synthase) and PGEs (Prostaglandin E synthase), wherein some of these effects are mediated by inhibition of NF-kB translocation.

Beneficial effects in clinical dogs with symptoms of OA are reported after treating the dogs with β1.3/1.6 glucans.

Resveratrol is a phenolic compound present in plants (e.g. wines, peanuts, mulberries, etc.). Such compound belongs to the group of compounds that protect plants from microbial infections. The relationship between this compound and OA has been found in limiting matrix loss and anti-apoptotic activity.

Berberine is a botanical-derived iso-quinoline alkaloid isolated from root of different plants with anti-inflammatory and antioxidant properties.

The inventors are now the first to establish that since insects such as in particular Black soldier fly, contain glucosamine, the presence of this glucosamine in e.g. BSF, BSF protein isolates or extracts or compositions derived therefrom or isolated therefrom, etc. (e.g. water-soluble BSF protein, hydrolysed BSF protein), provides these insects such as BSF or extracts or compositions derived therefrom or isolated therefrom, etc. with relevant bioactivity as an anti-arthritic composition based on said presence of glucosamine. That is to say, presence of glucosamine in BSF protein compositions or extracts or isolates or hydrolysates thereof endows such compositions with anti-arthritis activity, more in particular, anti-OA activity, such as in relieving inflammation and/or pain, or in preventing inflammation, pain, non-ability to freely move and use joints e.g. pain free.

Glucosamine and chondroitin sulphate serve as exogenous sources of building blocks for cartilage. In addition, recently, it has been established that also combined usage of low molecular weight chondroitin sulphate (LMWCS) and glucosamine can up-regulate chondrocytes and reduce cartilage degradation. Positive outcomes of this combined treatment for OA has been reported in humans and animals. Thus, the inventors are the first to establish the use of insect protein, for example BSF protein or hydrolysates thereof or water-soluble fraction thereof or hydrolysed water-soluble fraction thereof, in the treatment or prevention of arthritis such as OA, and/or in the treatment and/or prevention of disease symptoms such as inflammation and pain, etc. The protein is typically derived from insect larvae such as BSF larvae. Typically, such larvae are processed as part of a method to provide the protein.

Processing may encompass steps of washing the larvae (e.g. rinsing with water), mincing the larvae, heating, e.g. pasteurizing the (minced) larvae, extracting water-soluble protein from minced and/or pasteurized larvae, hydrolysing (water-soluble fraction of) isolated protein, e.g. after any one or more of washing, mincing and pasteurizing larvae such as BSF larvae, preferably minced and pasteurized BSF larvae.

Chitin is a large hydrophobic homo-polymer of B-(1-4)-linked N-acetyl-D-glucosamine synthetized by membrane-bound chitin synthase enzymes. The chitin of the BSF larvae is the dominant source of glucosamine in the BSF protein composition.

An aspect of the invention relates to a dog feed composition comprising the BSF larvae protein composition of the invention. An aspect of the invention relates to the use of such dog feed composition as a feed for a dog suffering from any one or more of: inflammation, for example inflammation in a joint affected by osteoarthritis, synovial inflammation and/or synovitis, such as in a joint affected by osteoarthritis, damage of structural biomolecules of the joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation and/or macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis. The dog does not suffer from OA, or suffers from OA.

The invention is further illustrated by the following examples, which should not be interpreted as limiting the present invention in any way.

### EXAMPLES & EMBODIMENTS

### Materials and Methods

### Raw Materials

Chicken meal (CM) was purchased from an online webshop in October 2020. Three types of BSF protein derivatives namely: pasteurized minced meat supplied frozen at −20° C.; enzymatically hydrolyzed and pasteurized minced meat also supplied frozen at −20° C.; and hydrolysate of water-soluble proteins supplied as dry powder were provided by Protix B.V. (Dongen, The Netherlands) in November 2020. Product composition, storage conditions and method employed to develop water-soluble extract were similar or the same as indicated in Mouithys-Mickalad et al. and in international application WO 2021/054823 A1.

Live and washed larvae (black soldier fly) of 14 days old post hatching (5 kg) were collected just before being subjected to mincing by using the mincer and subsequently processed or first stored at 4° C. until used. For each experiment 100 g larvae were minced freshly. Hundred g of minced larvae were heated to 90° C. (with continuously stirring) and the product was kept at this temperature for 80-120 s. In particular, protein is obtained when minced black soldier fly larvae are heated for 80 seconds at 90° C. The insect pulp (puree), i.e. BSF pasteurized minced meat, obtained was cooled to 3° C.-7° C. More in detail, Fourteen days old BSFL (black soldier fly larvae) were washed with tap water and then immediately minced using a blender. Then, samples were pasteurized using the micro-cooker for 80 seconds at 90° C. Samples were placed in the fridge (4° C.), therewith providing cooled heated insect pulp.

### Protein Hydrolysis

A puree of BSF larvae, also referred to as BSF PureeX™ (BSF-P), and hydrolyzed puree (BSF-HP) obtained by enzymatic hydrolysis of the puree of BSF larvae, were prepared by Protix B.V. (Dongen, The Netherlands). The puree and the enzymatically digested puree were obtained according to the following method. Live and washed Black Soldier Fly larvae of 14 days old (after hatching) were collected just before being subjected to the mincer for mincing the larvae (therewith providing larvae pulp (also referred to as puree)), and subsequently stored at 4° C. until used. For each experiment, larvae were minced freshly. The minced larvae was treated with 0.1% or 0.5% Flavourzyme, based on the mass of the minced larvae, for 0.5-3 hours, for example 1 to 2 hours, at 45° C. to 65° C. (±2° C.) under continuous stirring. The batch of hydrolysed BSF larvae puree applied was obtained by enzymatic hydrolysis for 1 hr at 50° C. (±2° C.). Flavourzyme (Novozymes, Denmark) is a combination of aminopeptidases which have endopeptidase and exopeptidase activities. The enzyme-treated minced larvae, and control larvae without enzyme treatment, were heated to 90° C. and the product was kept at this temperature for 80 seconds. The obtained insect puree were used for measuring free amino acid content (Free amino acid content (n=1)—Tested in puree) and pepsin digestibility (Digestibility (n=1)—Tested in puree). Protein meal was obtained from the heated BSF larvae pulp, and from the two batches of heated BSF larvae pulp that was first subjected to enzymatic hydrolysis of proteins using either 0.1 wt %, or 0.5 wt % Flavourzyme prior to the heating step at 90° C., e.g. by steps of protein separation from the pulp, evaporation, drying and grinding. In all experiments performed, a dose dependent effect was observed, when the amount of applied enzyme is considered. Thus, enzymatic treatment of minced larvae using Flavourzyme prior to the heating step increases the free amino acid content and increases pepsin digestibility of the obtained hydrolysed larvae puree, and as a result, the obtained puree comprising hydrolysed protein has a better taste (free amino-acid content relates to attractive, appealable taste when animals and humans consume a product comprising free amino-acids), is highly digestible and has anti-oxidant properties (see test results, here below). In addition, enzymatic treatment of BSF larvae pulp (pure; minced larvae) improves the fat separation in the following separation step after enzymatic hydrolysis and heat-treatment of the enzymatically digested pulp, which increases the fat extraction from the protein meal, when compared to fat separation from the protein fraction obtained with larvae puree that was not subjected to an enzymatic hydrolysis step prior to heating at 90° C.

### Raw Materials

Chicken meal (CM) was purchased from an online webshop in September 2019. The chemical composition of both ingredients as declared by the supplier is indicated in table 1.

TABLE 1
Chemical composition of chicken meal
(as in basis, provided by supplier).

| Nutrients | Chicken meal |
|---|---|
| Moisture (g/kg) | 60.0 |
| Crude protein (g/kg) | 700.0 |
| Crude fat (g/kg) | 120.0 |
| Added antioxidant | No |
| Form | Powder |

BSF-P and hydrolyzed puree BSF-HP were prepared by Protix B.V. (Dongen, The Netherlands). (1). BSF-P was pasteurized BSF minced ‘meat’ (puree, pulp) supplied frozen at −20° C. BSF-P is also the raw material to produce BSF protein meal (ProteinX™). (2). BSF-HP was hydrolyzed and pasteurized BSF meat (puree) also supplied frozen at −20° C. (3). The chemical composition of the two ingredients BSF-P and BSF-HP were as is indicated in table 2.

TABLE 2
Chemical composition of BSF protein derivatives heated puree and
heated hydrolysed puree (as in basis, provided by supplier).

| Nutrients | BSF-P¹ | BSF-HP² |
|---|---|---|
| Moisture (g/kg) | 700.0^{a} | 700.0^{a} |
| Crude protein (g/kg) | 120^{a} | 120^{a} |
| Crude fat (g/kg) | 122.5^{a} | 122.5^{a} |
| Added antioxidant | No | No |
| % of total proteins <1000 Da | >6 | >24 |

| | |
|---|---|
| Form | Frozen minced meat |

¹BSF-P: BSF PureeX ™;
²BSF-HP: BSF hydrolyzed puree;
^{a}Mean values based on the range established at Protix.

Water soluble extracts were prepared for CM, BSF-P and BSF-HP. These products (100 g each) were dissolved with six times volumes of Milli-Q water based on their respective dry matter contents (e.g. BSF-P had dry matter content of 33.3% and was diluted 200 ml Milli-Q water) and stirred for 2 h on a magnetic stirrer. Post centrifugation (1000×g for 30 min at 4° C.), the top fat layer was removed and the supernatant was filtered using Whatman Filter (grade 4). The centrifugation and filtration step was repeated again to remove all non-soluble residues. Finally the supernatant was filtered using a Sterlip Filter (50 mL, 0.22 μm) and freeze dried over a period of two days to obtain respective water soluble extract powders. All four water soluble extract were stored in a desiccator (at 18° C.) until further use.

### Protein Quantification

Total protein content of the four water soluble extracts was analysed using Bicinchoninic acid (BCA) protein assay (Smith et al., 1985). The calibration curve was obtained using bovine serum albumin (BSA) as standard at concentrations: 0, 0.125, 0.25, 0.5 and 1 mg/ml. Stock solutions of 3 mg/ml water soluble extracts were used for analysis. A test solution was made by dissolving 4900 μl BCA (49/50) and 100 μl copper (II) sulfate (1/50). Sample stock solutions (10 μl) and test solution (200 μl) were added in wells of 96-well plate. This plate was incubated at 37° C. for 30 min and absorbance was measured at 450 nm using a Multiscan Ascent (Fisher Scientific, Asse, Belgium).

The calibration curve obtained using BSA, equation of the line and R-square value are established. The optical density of samples and relative concentration of proteins (calculated using equation of line) are mentioned in table 3. BSF-P extract solution (3 mg/ml) exhibits the highest, on the other hand BSF-HP solution exhibits the lowest protein concentrations amongst the tested solutions using Bichinchoninic acid assay.

TABLE 3
Protein quantification using Bichinchoninic acid assay

| | | Mean | Protein |
|---|---|---|---|
| | Product used for testing | optical | concentration |
| Product | in all the assays | density | (mg/ml) |
| BSF-P¹ | Water soluble extract | 0.486 | 1.013 |
| BSF-HP² | Water soluble extract | 0.365 | 0.648 |
| CM³ | Water soluble extract | 0.481 | 0.998 |

¹BSF-P: BSF PureeX ™;
²BSF-HP: BSF hydrolyzed puree;
³CM: Chicken meal.

### Dry Matter & Moisture Content

To determine the dry matter content, first, the moisture content was determined in accordance with EC-152/2009. Then, dry matter was calculated by subtracting the moisture content from the initial mass of the sample.

An aqueous water-soluble protein composition or a dried water-soluble protein composition wherein the water-soluble proteins are substantially completely dissolvable in an aqueous solution such as water, is provided by isolating a proteinaceous fraction from black soldier fly larvae according to the method as described in European patent application EP2953487, in the Examples section, Example 1, page 12, line 8-13 and page 13, line 3-5. In brief, larvae of black soldier fly were provided and subjected to the method to convert insects or worms into nutrient streams, as substantially outlined here below:

Method to convert insects or worms into nutrient streams, comprising the steps of:
(a1) providing larvae of black soldier fly
(a2) reducing the insects in size,
(a3) obtaining a pulp from insects, then
(b) heating the pulp to a temperature of 90° C. for 80 seconds, and then
(c) subjecting the heated pulp to a physical separation step thereby obtaining a fat fraction, an aqueous protein fraction (referred to as “larvae water”) and a solid-containing fraction.

The aqueous protein fraction is an aqueous water-soluble protein fraction when black soldier fly larvae are subjected to the method to convert insects into nutrient streams. The aqueous water-soluble protein fraction is in some embodiments dried after step (c) using spray-drying, therewith providing dried black soldier fly larvae proteins. The method does not comprise enzymatic treatment of the pulp in any of the steps of the method. Optionally, the method does comprise enzymatic treatment of the larvae pulp, though for the current example, no enzymatic digestion steps were applied in the method to convert black soldier fly larvae into nutrient streams. In step (b) of the method, the minced black soldier fly larvae are pasteurized by heating the pulp (or ‘puree’) at 90° C. for 80 seconds, therewith providing pasteurized ‘meat’ of larvae. The pasteurized meat is subsequently in step (c) mechanically separated to obtain the liquid protein fraction (larvae water). The aqueous protein fraction is either used directly ‘as is’ without further treatment steps (for example drying or concentration) before provided as aqueous insect-protein composition comprising at least one protein in step (a) of the method of the invention for the provision of enzymatically hydrolysed insect-proteins, or the aqueous protein fraction (larvae water) is first concentrated, for example three to twelve times, such as 5-10 times, or first dried for example using spray-drying, before being subjected to dissolving in an aqueous solution such as water, and then provided in step (a) of the method of the invention as aqueous insect-protein composition comprising at least one protein.

The crude protein content of the larvae water obtained with step (c) of the here above outlined method to convert insects into nutrient streams, was 3.8% by weight based on the total weight of the larvae water. The crude fat content was 0.3% by weight based on the total weight of the larvae water. For the obtained larvae water, the total plate count assessed as the aerobic mesophilic count at 30° C. (ISO 4833) was 26000 cfu/g; the Bacillus cereus count at 30° C. (ISO 7932) was <40 cfu/g; the Clostridium perfringens count at 37° C. (ISO 7937) was <10 cfu/g; the Escherichia coli count at 44° C. was <10 cfu/g; and Salmonella was not detected in 25 g of the product, using PCR fast method (ISO 6579). Thus, in the larvae water, the microbial count was less than 40 cfu/g protein for Bacillus cereus; less than 10 cfu/g protein for Clostridium perfringens; less than 10 cfu/g protein for Escherichia coli; and the Salmonella count was 0 cfu/g protein when 25 g of the larvae water was assessed. Herewith, the microbial count was within value boundaries that should be reached for application of the larvae water in food products or food ingredients.

The aqueous water-soluble protein fraction (larvae water), either or not concentrated, or first dried and then dissolved again, is applied as the substrate for enzymatic hydrolysis of the at least one water-dissolvable protein in black soldier fly larvae water-soluble protein fraction. The liquid aqueous water-soluble protein fraction contains approximately 91% moisture content by weight, about 4% proteins by weight based on the total weight of the aqueous protein fraction (larvae water), and the liquid aqueous protein fraction had low fat content (<1% by weight based on the total weight of the aqueous protein fraction, i.e. 0.3% for the current preparation). The larvae water is a stock solution of dissolved water-soluble proteins that does not need any dilution step before enzymatic hydrolysis of the water-soluble proteins. The aqueous water-soluble protein fraction (larvae water) does not comprise water-insoluble chitin.

### Enzymatic Hydrolysis

The aqueous water-soluble protein fraction (larvae water) was subjected to enzymatic hydrolysis in a bioreactor with temperature control (30° C. to 100° C.), pH control (pH is between 4 and 9) and with continuous stirring (up to 1250 rpm). The proteins were enzymatically hydrolysed in some examples using a single amino-peptidase and in further examples using a combination of aminopeptidases which have endopeptidase and exopeptidase activities. The enzyme concentration was 0.1% to 2% by weight based on the total weight of the aqueous water-soluble protein fraction comprising the enzyme(s), for the one or more amino-peptidases. For example, Flavourzyme (Novozymes, Denmark) was used at 1% by weight based on the total weight of the aqueous protein fraction comprising the enzyme(s).

Similarly, minced and heated larvae, i.e. puree, was hydrolysed. Optionally, the water-soluble protein fraction was extracted from the hydrolysed puree.

### Reaction Conditions

During enzymatic hydrolysis, the pH (typically between 4 to 8), the reaction temperature (typically from 35° C. to 60° C.) and the enzymatic hydrolysis time (typically from 2 hours to 12 hours) of the reaction depended on the type of selected enzyme(s). With the Flavourzyme, the aqueous water-soluble protein fraction was hydrolysed at pH 7 (which was also the pH of the larvae water), at a temperature of 50° C. during 6 hours.

### Heating to Induce Enzyme Deactivation

The enzymatic hydrolysis reactions were terminated by heat deactivation of enzyme(s) at 75° C. to 110° ° C. for 1 minute to 10 minutes. Commonly, the enzymatic hydrolysis reaction was terminated by heating the reaction mixture of the aqueous water-soluble protein fraction comprising the enzyme(s), at 100° C. for 2 minutes, providing enzymatically hydrolysed black soldier fly larvae water-soluble proteins. When the Flavourzyme enzymes were applied, the enzymatically hydrolysed proteins are referred to as Hydrolysed Insect Extract 1 (“HIE1”, or “HIE 1”).

The chemical compositions of the enzymatically hydrolysed black soldier fly larvae water-soluble proteins HIE 1 are outlined in Table 4. In addition, the chemical composition of the aqueous water-soluble protein fraction of black soldier fly larvae (larvae water) is provided in Table 4. The free amino acid compositions of the aqueous water-soluble protein fraction of black soldier fly larvae (larvae water) and HIE 1 are outlined in Table 5. While counts of some pathogenic microbes is mentioned in table 6.

TABLE 4
Chemical compositions of the aqueous water-soluble protein fraction
of black soldier fly larvae (larvae water), the enzymatically
hydrolysed black soldier fly larvae proteins HIE 1

| | Aqueous water-soluble protein | |
|---|---|---|
| | fraction of black soldier fly | |
| Component^{‡} | larvae (larvae water) | HIE 1 |
| Moisture | 90.9% | 90.8 |
| Proteins | 3.8% | 3.8% |
| Lipids | 0.3% | <0.3% |
| Ash | 1.2% | 1.3% |
| Starch | 0.8% | <0.5% |
| Total sugars | 0.9% | 1.5% |
| (including glucose) | | |

^{‡}the amounts of the listed components are provided as the weight percentage based on the total weight of the analysed compositions.

TABLE 5
Free amino acid composition of the enzymatically hydrolysed
black soldier fly larvae proteins HIE 1. The amounts
of the free amino acids is determined by applying
the DJA75 test (ISO 13903: 2005/IC-UV).

| Component | HIE 1 |
|---|---|
| Pepsin Digestibility | 100% |
| Proteins <1000 Da | 100% |
| (weight % of total weight of proteins) | |
| Free amino acid content (weight % of | 47.7% |
| total weight of proteins) | |
| Free Alanine^{‡} | 0.272% |
| Free Arginine | 0.171% |
| Free Aspartic acid | 0.0740% |
| Free Cysteine | 0.0180% |
| Free Glutamic acid | 0.137% |
| Free Glycine | 0.0860% |
| Free Histidine | 0.0940% |
| Free Isoleucine | 0.0620% |
| Free Leucine | 0.0940% |
| Free Lysine | 0.116% |
| Free Methionine | 0.0120% |
| Free Phenylalanine | 0.0560% |
| Free Proline | 0.172% |
| Free Serine | 0.0700% |
| Free Threonine | 0.0850% |
| Free Tryptophane | 0.0360% |
| Free Tyrosine | 0.128% |
| Free Valine | 0.144% |
| Total free amino acids | 1.81% |

^{‡}the amounts of the listed free amino-acid residues are provided as the weight percentage based on the total weight of the protein hydrolysate, the protein hydrolysates comprising 4% by weight protein based on the total weight of the protein hydrolysate compositions. The amount of free amino acid residues of the total protein content of HIE 1 is provided as a weight percentage based on the total weight of the protein content of HIE 1.

More than fifty percent by weight of the hydrolysed proteins, based on the total weight of protein present in HIE 1 (i.e. 100%), are detected as very short chain peptides. “Very short chain peptides” is herein defined as peptides having an amino-acid residues chain length of between about 6 amino acid residues and about 20 amino acid residues.

TABLE 6
Counts of pathogenic bacteria in the enzymatically
hydrolysed black soldier fly larvae proteins HIE 1

| Microbes cfu/g | HIE 1 |
|---|---|
| Total plate count | 28000 cfu/g |
| E. coli | <10 cfu/g |
| Salmonella | Not detected in 25 g |
| B. cereus | <10 cfu/g |
| C. perfringens | <10 cfu/g |

The total plate count, also referred to as ‘Aerobic Mesophylic Count 30° C.’ (equivalent to ISO 4833), was determined by Nutrilab (Rijswijk, NL); the Bacillus cereus count was assessed at 30° ° C. (equivalent to ISO 7932); the Clostridium perfringens count was determined at 37° C. (equivalent to ISO 7937); the Escherichia coli plate count was assessed at 44° C.; The Salmonella count was assessed using PCR fast method (equivalent to ISO 6579). Thus, in the product HIE 1, the microbial count was less than 10 cfu/g protein for Bacillus cereus; less than 10 cfu/g protein for Clostridium perfringens; less than 10 cfu/g protein for Escherichia coli; and the Salmonella count was 0 cfu/g protein when 25 g of the HIE 1 was assessed. Herewith, the microbial count for HIE 1 was within value boundaries that should be reached for application of the larvae water in food products or food ingredients. That is to say, according to the European Commission, in “OECD issue paper on microbial contaminants limits for microbial pest control products”, the detected plate counts for the indicated microbes was within acceptable limits according to the European Commission guidelines.

### Glucosamine Content

Glucosamine content analysis of BSF protein derivatives (P, HP, AHP) and chicken meal (CM) was performed by Eurofins Food Testing B.V. (Barendrecht, The Netherlands). Samples were mixed with phenyl isothiocyanate in a pre-column derivatization reaction. Following this, samples were separated on a reverse phase ultra-liquid chromatography system equipped with an ethylene bridged hybrid column. Quantification of peaks was done against an external standard: dog food comprising insect protein (“Insect Protein All Breeds Dog Food”, Yora, Warninglid, UK).

As said, glucosamine content was measured in BSF protein derivatives as well as chicken meal. For biochemical investigations, hydrolysate of water-soluble proteins (APH) was used as it is, because of high water solubility. Whereas, in case of other raw materials (other two BSF protein derivatives and chicken meal), because of limited water solubility respective water-soluble extracts were used for testing.

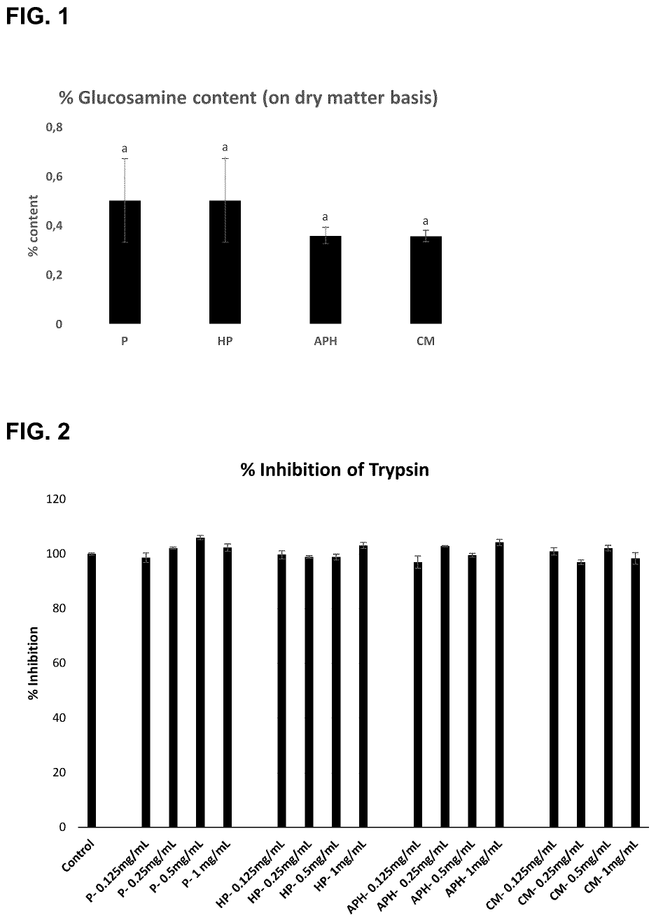

The glucosamine contents of P, HP, APH and CM is indicated in FIG. 1 and in Table 7. There were no significant differences in the glucosamine content amongst the samples (p<0.05).

TABLE 7
Glucosamine content of BSF protein derivatives and chicken meal

| | Glucosamine content (weight |
|---|---|
| Component | % based on dry matter) |
| Pasteurized minced meat of BSF | 0.5 ± 0.2^{a} |
| Hydrolyzed and pasteurized minced | 0.5 ± 0.2^{a} |
| meat of BSF | |
| Hydrolysate of water-soluble BSF | 0.4 ± 0.0^{a} |
| proteins | |
| Chicken meal | 0.4 ± 0.0^{a} |

Data is presented as mean±standard deviation (n=3). Letter above the bars represent significant differences (p<0.05). a: weight percentage (on dry matter basis) compared to the total mass of the (hydrolysed) protein composition.

Glucosamine and its salts are commonly used as nutraceutical supplements to ease the pain in dogs suffering with OA. Glucosamine, being an amino monosaccharide, is the preferred substrate for the biosynthesis of glycosaminoglycan, which is further used for the biosynthesis of proteoglycans that form cartilage. Three insect protein samples tested contain 0.4 to 0.5% by weight glucosamine (on dry matter basis) in the monomeric form. Therefore, dry pet food containing 30% of these insect proteins can supply about 120 mg glucosamine per 100 g of formula. A popular pet food brand currently recommends feeding 250 to 340 g insect-based formula per day to dogs with body weight 20 to 30 kg. This feeding pattern will render about 300 to 400 mg glucosamine to consuming dog, which is considerable when compared to commercial glucosamine supplements available in market that contain 300 to 1600 mg glucosamine. However, there are some reports that provide evidence about limited uptake of orally administer glucosamine in dogs. For proper functioning of the activity against OA, the in vivo uptake of glucosamine present in BSF proteins is essential.

### Proteinase Inhibitory Assay

Proteinase inhibition ability of water-soluble extracts of CM, P, HP and APH (i.e. hydrolysate of water-soluble BSF proteins (APH), pasteurized minced meat of BSF water-soluble extract (P), hydrolyzed and pasteurized minced meat of BSF water-soluble extract (HP), chicken meal water-soluble extract (CM)) was evaluated using the protocol of Murugesan et al. Reaction mixtures were obtained by mixing 1 ml of 25 mM tris-HCl buffer and 0.06 mg trypsin with 1 ml water-soluble extracts (at 0.125, 0.25, 0.5 and 1 mg/ml). This was followed by incubation for 10 min at 37° C., addition of 1 ml azocasein solution (0.8% w/v) and final incubation for 20 min. Then 2 ml of perchloric acid (70%) was added to the mixtures for arresting the reaction. The resulting mixtures were centrifuged, and absorbance of respective supernatants were measured at 280 nm wavelength. Inhibition activities (%) were calculated using following formula:

[(absorbance of control−absorbance of respective sample)/(absorbance of control)]×100%.

The % inhibition of trypsin during hydrolysis of azocasein by addition of water-soluble extracts from P, HP, APH and CM (i.e. hydrolysate of water-soluble BSF protein (APH), pasteurized minced meat of BSF extract (P), hydrolyzed and pasteurized minced meat of BSF extract (HP), chicken meal extract (CM)) is indicated in FIG. 2. All the water-soluble extracts used during this study did not show any trypsin inhibition activity during this study.

Development of OA occurs in three distinct phases: (a). phase 1—proteinase mediated hydrolysis of cartilage matrix; (b). phase 2—fibrillation and disintegration of cartilage surface that is coupled with release of disintegration products into synovial fluid; and (3). phase 3—engulfing of disintegration products by synovial cells (via phagocytosis) and production of proteinase (go back to phase 1) and cytokines that initiate inflammation. Metalloproteases and serine proteinase are considered to have key role during hydrolysis of cartilage matrix. The inventors used trypsin, which is a serine proteinase to evaluate the proteinase inhibition activity of BSF protein derivatives and chicken meal. None of the tested samples were able to arrest trypsin activity (see FIG. 2). Indicating that BSF protein derivatives will have no role in preventing OA during phase 1.

### Cell Membrane Stability Assay—Erythrocyte Stability Assay

Cell membrane stability assay was realized according to the protocol of Karimi et al. Blood used for the assay was extracted from a healthy horse in 10 ml tubes containing EDTA. It was centrifuged for 10 min at 3000 rpm and plasma supernatant was discarded. Obtained erythrocytes (red blood cells) were washed two times with 5 ml PBS solution at pH 7.4. Red blood cell pellets were diluted 10 times by phosphate buffer solution. Aliquots (0.5 ml) of this cell suspension were transferred to 5 ml tubes. This was followed by addition of 0.35 ml of 0.15 M phosphate buffer, 0.1 ml water-soluble extract solution of CM, P, HP and APH (at 0.25, 0.5, 1 and 2 mg/ml) and incubation for 10 min. The red blood cells (1 ml of above solution) were challenged by addition of 0.05 ml 25 mM 2,2′-Azinobis(2-amidopropane) di-hydrochloride (97%). For control, only buffer solution was used instead of water-soluble extract solutions. The resulting mixtures were incubated for 30 min at 57° C. Finally, the mixtures were chilled to 4° C. and centrifuged for 10 min at 3000 rpm. The supernatants (0.5 ml) were diluted with PBS and the absorbance was measured at 560 nm. Percentage inhibition toward lysis from 2,2′-Azinobis(2-amidopropane) di-hydrochloride (AAPH) was estimated using the formula:

[(absorbance of control−absorbance of respective sample)/(absorbance of control)]×100%.

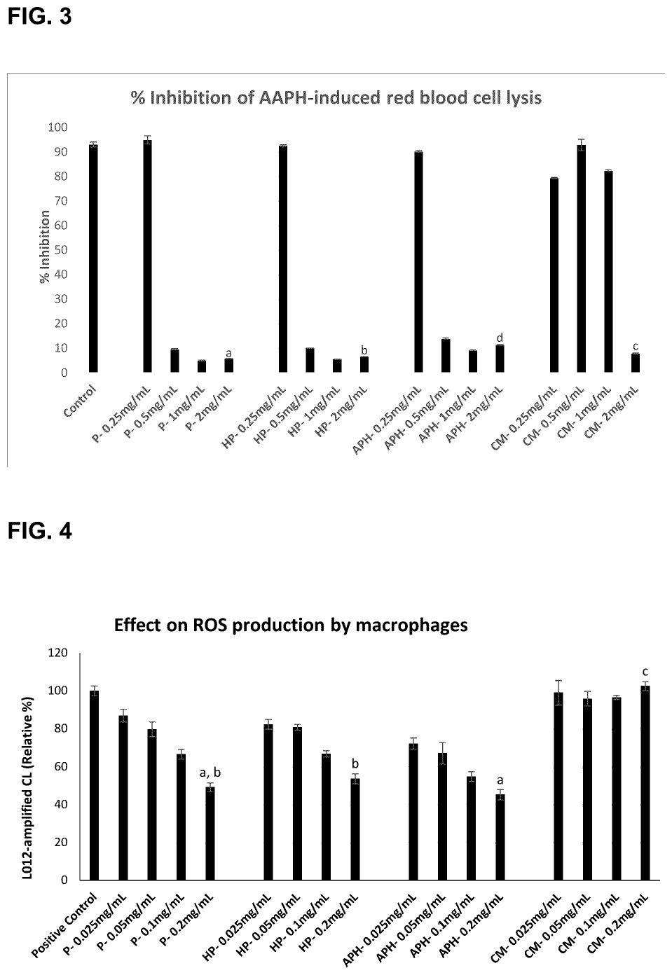

The protective effect of P, HP, APH and CM against free radical—(in this case AAPH) induced red blood cell lysis is indicated in FIG. 3. During this assay P, HP and APH showed an increase in inhibition with increasing concentration from 0.25 to 1 mg/ml. At the highest concentration used, % inhibition was in following order: P>HP>CM>APH (p<0.05). These results identified pasteurized meat of BSF larvae as a most effective composition in protecting the red blood cells against AAPH induced cell lysis with regard to all the tested samples.

Erythrocyte stability is crucial during arthritis. Literature published during last decade has highlighted the importance of erythrocytes in OA. There is already evidence showing the increase of erythrocyte sedimentation rate during OA. The increase of erythrocyte sedimentation rate could be attributed to the assault of ROS produced during the third phase of OA process. Furthermore, if the concentration of viable erythrocytes in the joint cavity decreases, it can have severe implications to the cartilage and synovial tissues. During this assay AAPH was used to generate ROS. AAPH is commonly used in biochemical assays to investigate cyto-protective effects of amino residues. AAPH can generate free radicals via spontaneous decomposition (at body temperature i.e., 37° C., when e.g. human subjects and dogs are considered). These free radicals can react with oxygen to produce ROS, which can further react with lipids present in cellular membrane to form peroxyl radicals. This process not only results in production of peroxyl radicals that participate in inflammatory process, but also results in disintegration of cells (including erythrocytes). Molecules that can stabilize the reactive products generated by AAPH are known to have cyto-protective effects and could contribute towards prevention of OA.

Surprisingly now, the inventors demonstrated that BSF protein derivatives have strong cyto-protective activity at all the concentrations ≥0.5 mg/ml (see FIG. 3). Previously, the inventors demonstrated the strong potential of BSF protein derivatives to donate hydrogens atoms and electrons in stabilizing free radicals (Mouithys-Mickalad et al., 2020). Indicating that BSF protein derivatives could instantly donate these chemical species to stabilize the intermediates of AAPH induced oxidation resulting in cyto-protective effects in e.g. human OA patients and dogs suffering from OA or even in human subjects and dogs in order to prevent OA (prophylaxis). At highest concentration, out of all tested samples BSF-P (pasteurized minced meat of insect) exhibited maximum inhibition of cell lysis.

### DPPH Assay

DPPH radical scavenging activity was analysed according to protocol of Brand-Willams et al., with some modifications. DPPH test solution was made by dissolving 10.5 mg DPPH in 40 ml ethanol. Test solution was made fresh and stored in dark until further use. DPPH working solution was made by diluting test solution with 10 times ethanol (to obtain absorbance of 0.6 to 0.8 at 517 nm). DPPH working solution (1920 μl) was mixed with 20 μl of samples dilutions (four water soluble extracts in Milli-Q water from minced and heated BSF larvae (puree) and from hydrolysed BSF larvae puree (BSF-HP) and from hydrolysed water-soluble protein extract from BSF larvae puree after mincing and heating larvae) to obtain final concentration of 0.0125, 0.025, 0.05, 0.1 and 0.2 mg/ml. The decrease in absorbance after 30 and 60 min of incubation in dark was recorded at 510 nm using HP 8453 UV-vis spectrophotometer (Agilent Technologies, Waldbronn, Germany). Instead of sample dilutions only Milli-Q water was used in case of control.

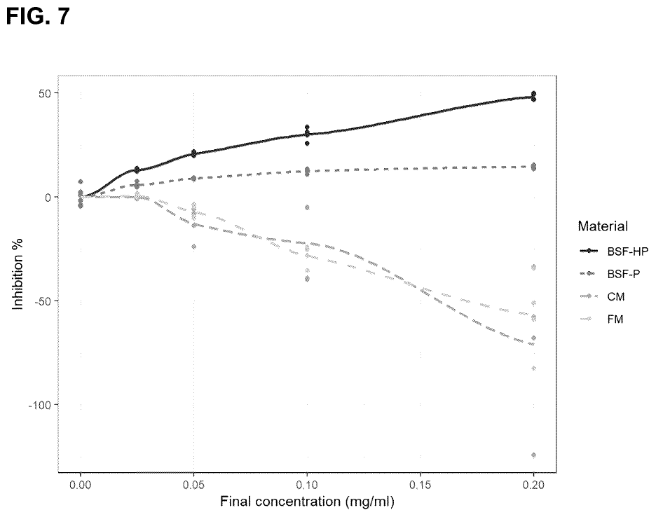

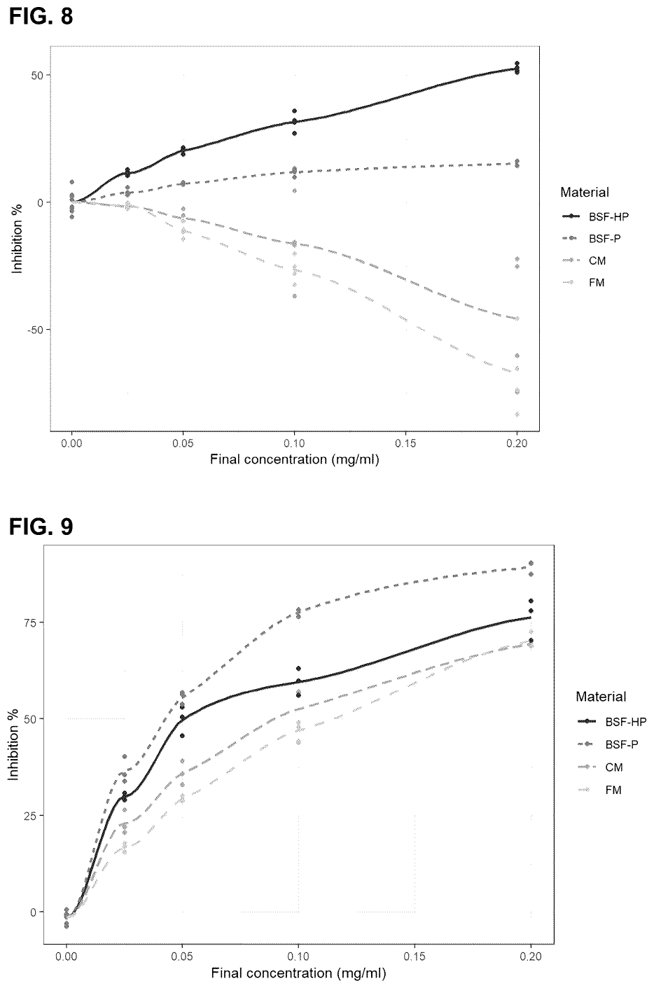

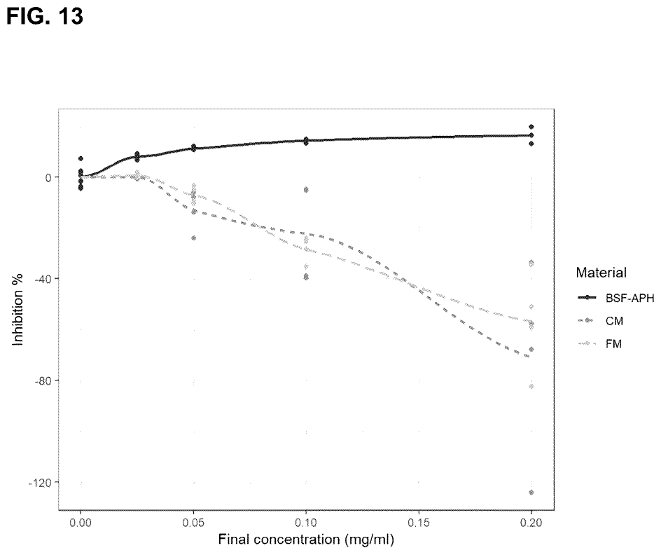

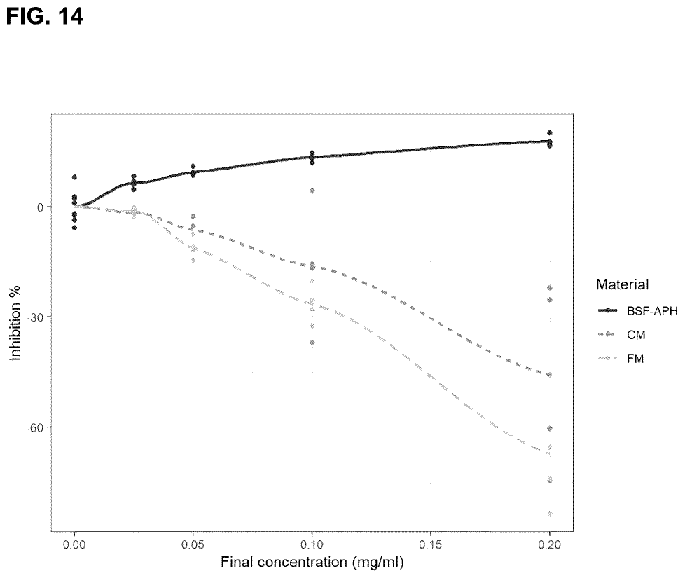

DPPH radical scavenging activity of all five samples after 30 and 60 minutes of incubation is indicated in FIG. 7 and FIG. 8, respectively, for BSF-P and BSF-HP, and in FIG. 13 and FIG. 14 for water-soluble protein hydrolysate (BSF-APH). The plot shows the measured values as well as fitted curves obtained from LOESS. CM exhibited pro-oxidant behavior at all tested concentrations after 30 as well as 60 minutes of incubation. The IC₅₀ of BSF-HP after 60 min of incubation is indicated in table A. It was not possible to calculate IC₅₀ for other samples (after 30 or 60 min of incubation) because the samples either exhibited pro-oxidant activity or 50% inhibition was not achieved during the assay. The Eₘₐₓ (maximum inhibition achieved during the assay) of all the samples are also indicated in table B and are in following order: BSF-HP>BSF-P and BSF-HP>BSF-P after 30 and 60 minutes of incubation, respectively. FM: fish meal control.

TABLE A
Antioxidant activity IC₅₀ (mg/ml) of samples
obtained using different assays

| Assay | BSF-P¹ | BSF-HP² | CM³ | BSF-APH |
|---|---|---|---|---|
| DPPH 30 min | NE^{c} | NE^{c} | PO^{d} | NE^{c} |
| DPPH 60 min | NE^{c} | 0.18 | PO^{d} | NE^{c} |
| ABTS 30 min | 0.04 | 0.05 | 0.09 | 0.03 |
| MPO^{a} SIEFED | NE^{c} | 0.14 | PO^{d} | 0.18 |
| MPO^{a} Classical | 0.10 | 0.09 | PO^{d} | 0.05 |
| CAA^{b} | 0.15 | 0.15 | NE^{c} | NE^{c} |

¹BSF-P: BSF PureeX ™;
²BSF-HP: BSF hydrolyzed puree; CM: Chicken meal; BSF-APH: BSF aqueous protein hydrolysate;
^{a}MPO: Myeloperoxidase;
^{b}CAA: Cellular antioxidant activity using neutrophil model;
^{c}NE: Not estimated because 50% inhibition was not achieved in tested concentrations;
^{d}PO: Not estimated because sample exhibited pro-oxidant activity on tested concentrations.

TABLE B
Antioxidant activity Eₘₐₓ (% inhibition) of
samples obtained using different assays

| Assay | Parameter | BSF-P¹ | BSF-HP² | BSF-APH | CM³ |
|---|---|---|---|---|---|
| DPPH 30 min | Emax (%) | 14.52 | 48.09 | 16.26 | PO^{c} |
| | C* (mg/ml) | 0.20 | 0.20 | 0.20 | — |
| DPPH 60 min | Emax (%) | 15.22 | 52.53 | 17.75 | PO^{c} |
| | C* (mg/ml) | 0.20 | 0.20 | 0.20 | — |
| ABTS 30 min | Emax (%) | 89.33 | 76.32 | 90.81 | 69.39 |
| | C* (mg/ml) | 0.20 | 0.20 | 0.20 | 0.20 |
| MPO^{a} SIEFED | Emax (%) | 36.23 | 77.58 | 53.08 | PO^{c} |
| | C* (mg/ml) | 0.20 | 0.20 | 0.20 | — |
| MPO^{a} Classical | Emax (%) | 89.66 | 83.82 | 90.86 | PO^{c} |
| | C* (mg/ml) | 0.20 | 0.20 | 0.20 | — |
| CAA^{b} | Emax (%) | 59.57 | 59.64 | 36.62 | 5.08 |
| | C* (mg/ml) | 0.20 | 0.20 | 0.20 | 0.20 |

*C: Concentration at which Eₘₐₓ is achieved;
¹BSF-P: BSF PureeX ™;
²BSF-HP: BSF hydrolyzed puree;
³CM: Chicken meal; BSF-APH: BSF aqueous protein hydrolysate;
^{a}MPO: Myeloperoxidase;
^{b}CAA: Cellular antioxidant activity using neutrophil model;
^{c}PO: Not estimated because sample exhibited pro-oxidant activity on tested concentrations.

DPPH and ABTS assays are commonly used to analyze antioxidant potential of food and feed products. DPPH radical scavenging activity represents the ability of a sample to donate hydrogen atom (referred as hydrogen atom transfer) or electrons (referred as single electron transfer) to stabilize free radicals. DPPH assay IC₅₀ and Eₘₐₓ for all tested samples are mentioned in table A and table B, respectively. Post 30 min of incubation, all the tested samples exhibit Eₘₐₓ <50% (with BSF-HP exhibiting highest Eₘₐₓ). On the other hand, after 60 min of incubation only BSF-HP exhibit Eₘₐₓ>50%. BSF—HP is manufactured by controlled hydrolysis of black soldier fly proteins and contains at least 24% of proteins <1000 Da. On the other hand BSF-P contains at least 6% proteins <1000 Da. The inventors were not able to find any representative literature for molecular weight distribution of the CM. However, according to the literature, CM contains 1.1% free amino acid (of total proteins) [Li, P.; Wu, G. Composition of amino acids and related nitrogenous nutrients in feedstuffs for animal diets. Amino Acids 2020, 1-20, doi:10.1007/s00726-020-02833-4.]. Which translates into CM containing at least 1.1% proteins <1000 Da. The capacity of proteinaceous materials to scavenge free radicals depends on the protein molecular weight distribution. Proteins with low molecular weight peptides could scavenge free radicals more efficiently. Free radical scavenging activity of proteinaceous molecules is also influenced by: (1). Amino acid composition: hydrophobic amino acids (for e.g. Tyr, Phe, Pro, Ala, His and Leu) have superior radical scavenging activity in comparison to hydrophilic amino acids; (2). Amino acid sequence: Peptides with amphiphilic nature could enhance radical scavenging activity of a sample. Chemical analyses have indicated that Tyr exhibit antioxidant behavior via hydrogen atom transfer mechanism. On the other hand, amino acids such as Cys, Trp and His exhibit antioxidant behavior via single electron transfer mechanism.

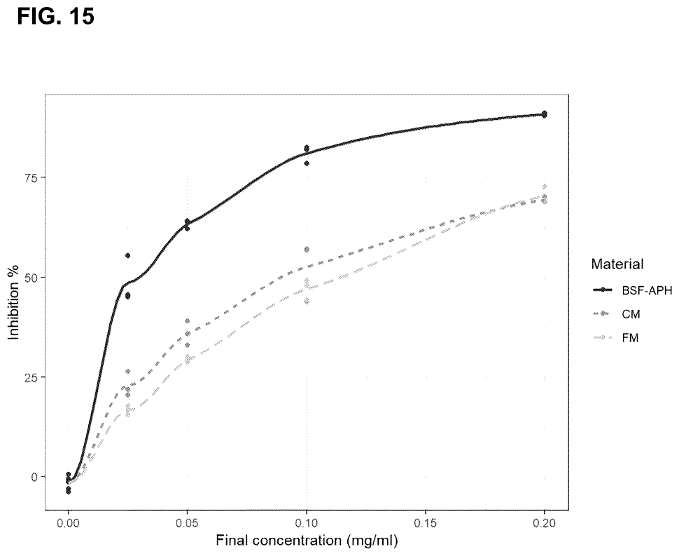

CM exhibits pro-oxidant behavior at most concentrations tested after 30 min as well as 60 min of incubation (see FIG. 7 and FIG. 8 for BSF-P and BSF-HP, and see FIG. 14 and FIG. 15 for water-soluble protein hydrolysate (BSF-APH)). This behavior mainly arises from the thermal processing. For CM, thermal processing commonly involves heating the raw product at high temperatures for 15 to 20 min. In Norway, during fishmeal production, wild caught fishes are subjected to heating at temperatures ≥70° C. for time ≥20 min in order to achieve 100 log 10 reductions of Enterobacteriaceae and Salmonella counts. Such strict thermal processing conditions may result in oxidation of fats and proteins. Fish meal contains lipids rich in polyunsaturated fatty acids that are more susceptible to thermal oxidation. Antioxidant are commonly added in fish meal to prevent the oxidation of polyunsaturated fatty acids. Heat induced oxidation of amino acids lead to development of wide range oxidation products. The pro-oxidant behavior of amino acid oxidation by products is already known. They can result in a wide range of health conditions in animal body. The black soldier fly protein derivatives (puree, hydrolysed puree) used and analyzed by the inventors were thermally processed at temperatures <100° C. for time <1.5 min (e.g. at 90° ° C. for 80 seconds). These thermal processing time-temperature combinations of the current invention were adopted to ensure minimum damage to nutrients (proteins and fat) and adequate inactivation of pathogenic microbiota. This implies that pro-oxidant behavior of CM arises mainly due to stringent production method. FM: fish meal control. Methods and results are according to the methods and results in Mouithys-Mickalad et al. (2020), WO 2021/054824 and WO 2021/054823.

### ABTS Assay

ABTS cation radical scavenging activity was analysed according to protocol of Arnao et al., with some modifications. ABTS test solution was made by dissolving 7.0 mmol/l ABTS and 2.45 mmol/l potassium persulfate in Milli-Q water. The test solution was kept overnight in dark at room temperature. ABTS working solution was made by diluting with methanol to obtain the absorbance between 0.7 and 0.8 at 734 nm. ABTS working solution (1920 μl) was mixed with 20 μl of samples dilutions (four water soluble extracts in Milli-Q water) to obtain final concentration of 0.0125, 0.025, 0.05, 0.1 and 0.2 mg/ml. The decrease in absorbance after 30 min of incubation in dark was recorded at 734 nm using HP 8453 UV-vis spectrophotometer (Agilent Technologies, Waldbronn, Germany). Instead of sample dilutions only Milli-Q water was used in case of control.

ABTS cation radical scavenging activity of samples after 30 minutes of incubation is shown in FIG. 9 for BSF-HP and FIG. 15 for BSF-APH (measured values as well as fitted curves obtained from LOESS). All the samples exhibited a similar inhibition pattern i.e., % inhibition increased as a function of increasing concentration. The IC₅₀ of samples are mentioned in table A and are in following order: CM>BSF-HP>BSF-P. Lower the IC₅₀, higher is the ABTS cation radical scavenging activity. The Eₘₐₓ (maximum inhibition achieved during the assay) of all the samples are indicated in table B and are in following order: BSF-P>BSF-HP>CM.

ABTS cation radical scavenging denotes the ability of sample to donate electron and stabilize free radicals. ABTS assay IC₅₀ of all samples are indicated in table A. They are in following order: CM>BSF-HP>BSF-P. The higher the IC₅₀, the lower the antioxidant activity. In this assay even CM exhibits antioxidant activity. It appears that CM extracts may be efficient where free radical(s) could be stabilized using single electron transfer mechanism. However, it still exhibits lower scavenging activity in comparison to the surprisingly high scavenging activity of BSF derivatives BSF larvae puree and hydrolysed BSF larvae puree. FM: fish meal control. The IC₅₀ of samples are mentioned in table A and are in following order: FM>CM>BSF-APH. Lower the IC₅₀, higher is the ABTS cation radical scavenging activity. The Eₘₐₓ (maximum inhibition achieved during the assay) of all the samples are indicated in table B and are in following order: BSF-APH>FM>CM.

Dependence of radical scavenging activity on protein molecular weight is already explained here above. At Protix the inventors established that BSF-P and BSF-HP have exactly the same amino acids composition. However, due to protein hydrolysis, the amount of proteins <1000 Da is higher in BSF-HP than in BSF-P. It is therefore somewhat surprising that the BSF-P IC₅₀ value was slightly lower in comparison to BSF-HP. This could be explained by the mechanism of hydrolysis. Enzymatic hydrolysis is achieved through exo- and endo-peptidase. Exopeptidase cleaves the terminal peptide bond, on the other hand endopeptidase cleaves the non-terminal peptide bond. In both cases the sequence of amino acids is altered. The radical scavenging ability of resulting peptides via single electron transfer is also dependent on the amphiphilic nature of proteinaceous molecules. It is possible that peptides in BSF-HP are less amphiphilic in nature that results into lower ABTS cation radical scavenging activity of BSF-HP compared to BSF-P.

The current inventors now surprisingly established that BSF-P already exhibits ABTS cation radical scavenging Eₘₐₓ of as high as 89, (at 0.2 mg/ml). This shows that fractioning BSF-P reveals fractions that have very strong antioxidant potential. FM: fish meal control. Methods and results are according to the methods and results in Mouithys-Mickalad et al. (2020), WO 2021/054824 and WO 2021/054823.

### Neutrophil Response Modulation Activity

Strong free radical scavenging activities of BSF derivatives are evident from the DPPH and ABTS assays. Furthermore, all the samples were also tested for neutrophil response modulation activity. Neutrophils are white blood cells present in animal body (including humans, pets, fishes, poultry and swine). They are involved in the primary defense against pathogens. When pathogenic microbes enter the animal body, neutrophils rush to the site of infestation and initiate defense. During granulation, neutrophil release a wide range of oxidative enzymes including NADPH oxidase, which is responsible for production of superoxide anion and by product (e.g. hydrogen peroxide). Superoxide anion can further react with nitric oxide radical to produce per-oxynitrite. This process also generates hydroxyl radical (by reaction of hydrogen peroxide with metal ion). This battery of oxidative reactions are crucial to the defense of the host animal. However, these ROS generated during host defense can react with enzymes, proteins, lipids, etc. of body cells and result in the development of different health conditions (for e.g. cellular ageing, cancer, etc.). The neutrophil assay conducted in this research determines the ability of proteinaceous molecules to scavenge ROS produced as a result of neutrophil activity. PMA was used to activate protein kinase C present in neutrophils, which results in production of NADPH oxidase responsible for catalyzing ROS production. ROS production in system is coupled with lucigenin amplified chemi-luminescence. Ability of proteinaceous sample to scavenge ROS (particularly superoxide anion) is marked by decreased chemi-luminescence.

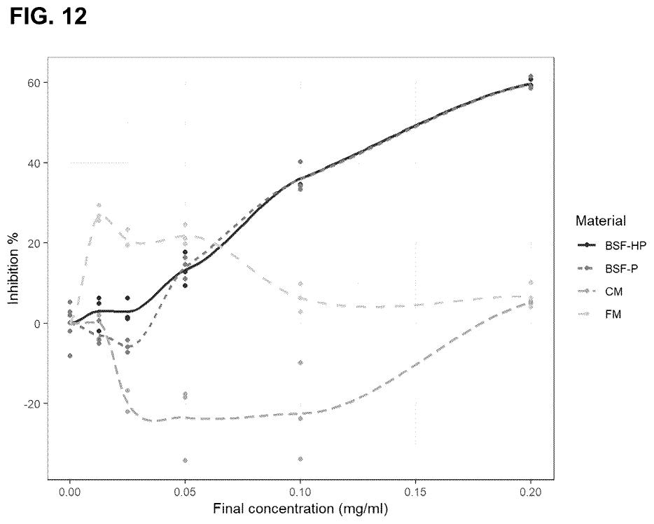

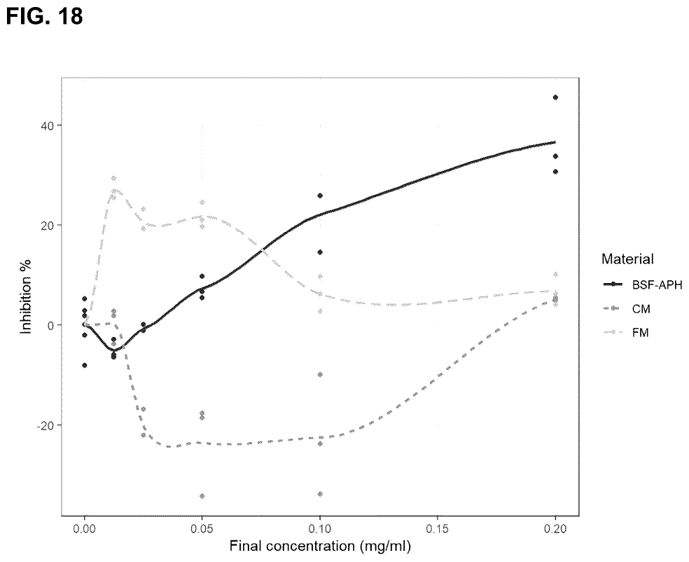

To the inventors knowledge, this is the first analysis of in vitro neutrophil response modulation activity of BSF derivatives BSF larvae puree and hydrolysed BSF larvae puree. CM exhibited pro-oxidant behavior at 3 out of 5 tested concentrations, and Eₘₐₓ of only 5% at 0.2 mg/ml (see FIG. 12, FIG. 18 and table B). CM is commonly used in pet food preparations. However, the comparative test results in the examples and embodiments of the invention indicate that CM inclusion offers little or no benefits relating to scavenging the ROS produced by neutrophils. Moreover, CM inclusion could even result in inflammatory damage to host cells. Repetitive inflammatory damage of canine or feline cells could translate into conditions such as accelerated aging, slow cognitive function, etc.

BSF-P exhibits Eₘₐₓ and IC₅₀ of 59.57% and 0.15 mg/ml, respectively (see table B). Moreover, BSF-HP also exhibits neutrophil response modulation activity comparable to BSF-P (see table A). FM: fish meal control. Methods and results are according to the methods and results in Mouithys-Mickalad et al. (2020), WO 2021/054824 and WO 2021/054823.

### Myeloperoxidase (MPO) Activity Using Specific Immunological Extraction Followed by Enzymatic Detection (SIEFED) Assay

SIEFED assay is a licensed method developed by Franck et al. for specific detection of animal origin MPO. MPO solution was made by diluting human MPO in 20 mM phosphate buffer saline (at pH 7.4), 5 g/l BSA and 0.1% Tween-20. Sample dilutions at final concentration of 0.0125, 0.025, 0.05, 0.1 and 0.2 mg/ml were incubated for 10 min (at 37° C.) with MPO solution at a final concentration of 25 ng/ml. After incubation, the mixtures were loaded into the wells of a 96 wells microtitre plate coated with rabbit polyclonal antibodies (3 μl/ml) against equine MPO and incubated for 2 h at 37° C. in darkness. After washing up the wells, the activity of the enzymes captured by the antibodies was measured by adding hydrogen peroxide (10 μM), NO2″ (10 mM) and Amplex™ Red (40 UM). The oxidation of Amplex™ Red into the fluorescent adduct resorufin was monitored for 30 min at 37° C. with Fluorosckan Ascent (Fisher Scientific, Asse, Belgium). Instead of sample dilutions only Milli-Q water was used in case of control.

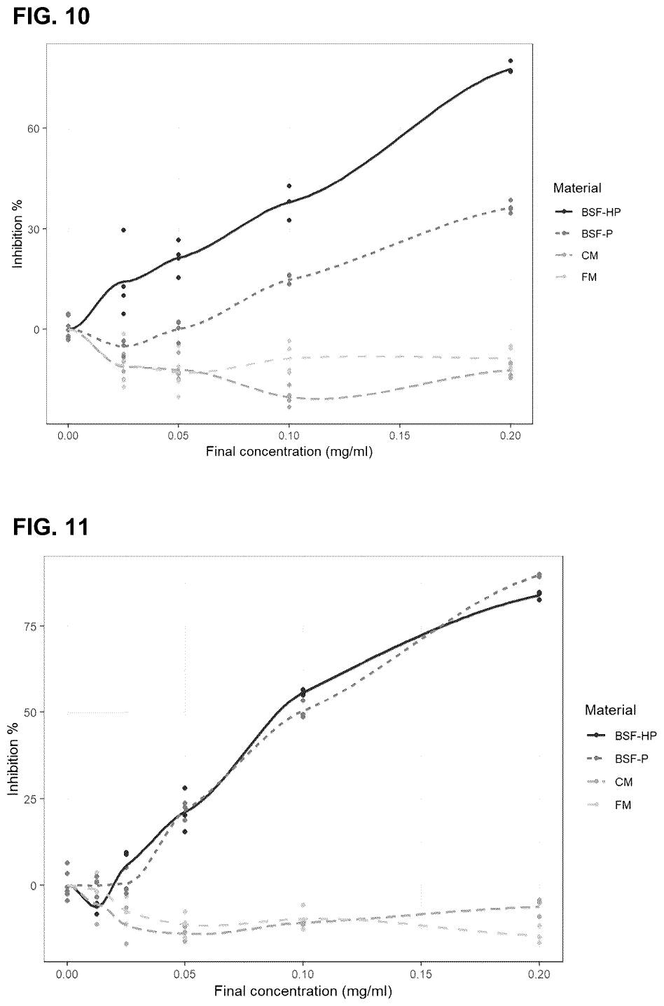

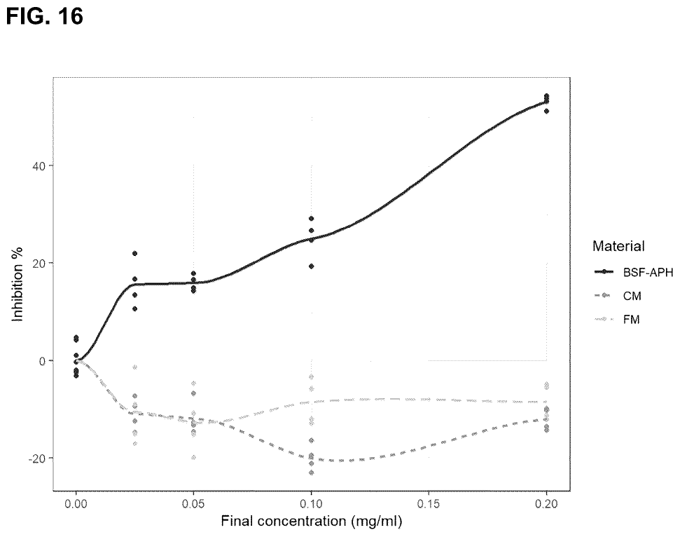

MPO response modulation activity of samples obtained using SIEFED assay is shown in FIG. 10 and FIG. 16 (measured values as well as fitted curves obtained from LOESS). BSF-HP exhibited strong inhibition behavior, with >75% inhibition at 0.20 mg/ml concentration. The IC₅₀ of samples are mentioned in table A and are in following order: BSF-HP. The Eₘₐₓ of samples are shown in table B, and are in following order: BSF-HP>BSF-P, and CM shows pro-oxidant behavior at all tested concentrations. On the other hand Emaz for BSF-P was <50%. FM: fish meal control. Methods and results are according to the methods and results in Mouithys-Mickalad et al. (2020), WO 2021/054824 and WO 2021/054823.

### Myeloperoxidase (MPO) Activity Using Classical Measurement

MPO solution was prepared as mention in section 2.6. Sample dilutions at final concentration of 0.0125, 0.025, 0.05, 0.1 and 0.2 mg/ml were incubated for 10 min (at 37° C.) with MPO solution at a final concentration of 25 ng/ml. After incubation, the mixture (100 μl) was immediately transferred into 96-well microtitre plate. This was followed by addition of 10 μl NO2 (10 mM) and 100 μl of Amplex™ Red and hydrogen peroxide mixture (at concentrations mentioned here above for the SIEFED assay). The oxidation of Amplex™ Red into the fluorescent adduct resorufin was monitored for 30 min at 37° C. with Fluorosckan Ascent (Fisher Scientific, Asse, Belgium) immediately after addition of relevation mixture. Instead of sample dilutions only Milli-Q water was used in case of control.

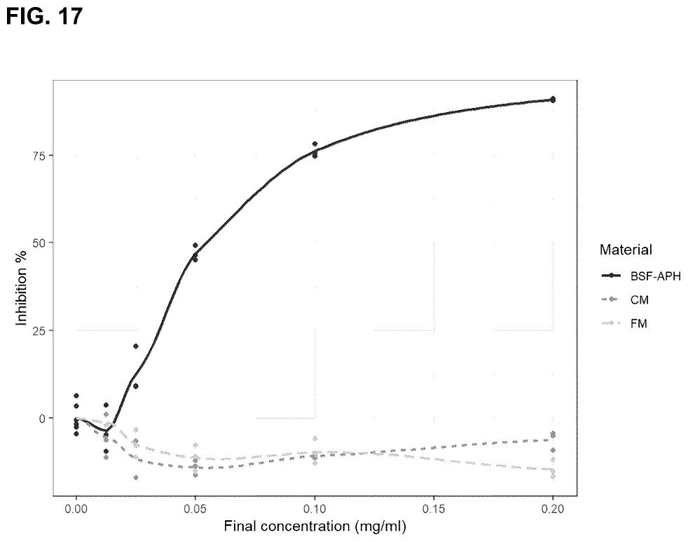

MPO response modulation activity of samples obtained using classical assay is indicated in FIG. 11 and FIG. 17 (measured values as well as fitted curves obtained from LOESS). CM exhibited pro-oxidant behavior at all tested concentrations. The Eₘₐₓ of all the samples tested are indicated in table B. BSF-P and BSF-HP exhibited Eₘₐₓ>75%. BSF-APH exhibited Eₘₐₓ>75%. The IC₅₀ of samples are mentioned in table A and are in following order: BSF-P>BSF-HP. FM: fish meal control. Methods and results are according to the methods and results in Mouithys-Mickalad et al. (2020), WO 2021/054824 and WO 2021/054823.

### Results: MPO Response Modulation Activity (SIEFED and Classical Assay)

The general mechanism of neutrophil response is known. The neutrophil extracellular trap contains several molecules required to inactivate pathogenic microbes. MPO enzyme present in neutrophil extracellular trap can produce hypochlorous acid from hydrogen peroxide and chloride ion. Additionally, MPO is capable of oxidizing tyrosine into the tyrosyl free radical. Both products of MPO oxidation (hypochlorous acid and tyrosyl free radical) are crucial to inactivate pathogens. Again, repetitive interaction of these molecules with animal cells result in inflammatory damage. In an animal body, MPO—Fe(III) (active form) reacts with hydrogen peroxide to form oxoferryl TT cation radical (Cpl form). Cpl form converts back into MPO—Fe(III) coupled with chloride ion transforming into hypochlorous acid. However, in the present experiment, back reduction of the Cp I form to MPO—Fe(III) was achieved in 2 stages. First reduction of Cpl to MPO—Fe(IV)=O via electron transfer through nitrite ions. Then, electron provisioning was done (via Amplex™ Red oxidation to resorufin reaction) which converts MPO—Fe(IV)=O to MPO—Fe(III) form. Proteinaceous molecules could prevent the oxidative damage resulting from MPO either by directly reacting with Cpl form and terminating the halogenation, or by donating hydrogen (hydrogen atom transfer) to ROS produced as a consequence of MPO activity. MPO response modulation activity was analyzed using the classical and SIEFED assay. The classical assay measures ability of sample to complex with Cpl form and stabilize ROS. Whereas in SIEFED assay, MPO is bound to rabbit polyclonal antibodies (and rest of the compounds are washed away), so it purely measures the ability of samples to complex with Cpl form.

As with neutrophil response modulation activity, MPO response modulation activity of BSF derivatives BSF larvae puree and hydrolysed BSF larvae puree is also being established by the inventors, after provision of the puree according to the method of the invention. CM exhibits pro-oxidant behavior in both the assays (see FIGS. 10 and 11, and see FIGS. 16 and 17). Presence of oxidative reaction products in CM (as a consequence of production process) that are capable of initiating pro-oxidative response has been already discussed in the section here above on DPPH radical scavenging activity. Detailed in vitro investigations realized by the inventors indicate that inclusion of CM in animal diets may result in inflammatory damage.

In classical assay, BSF derivatives exhibit surprisingly strong antioxidant potential, with IC₅₀ in following order: BSF-P>BSF-HP. In the SIEFED assay, BSF-P did not reach 50% inhibition (even at highest concentration tested). Thus while, BSF-P is more effective in stabilizing ROS, BSF-HP has higher efficacy in complexing with Cpl form of MPO. These observations show that the two BSF derivatives are suitable for use as an ingredient in human food, pet food and aquaculture formulations to effectively suppress inflammatory damages resulting from MPO activity. FM: fish meal control. Methods and results are according to the methods and results in Mouithys-Mickalad et al. (2020), WO 2021/054824 and WO 2021/054823.

BSF protein derivatives offer anti-oxidative advantage over CM.

### Cellular Antioxidant Activity

Preparation of the neutrophil and phorbol 12-myristate 13-acetate (PMA) solutions were made according to Paul et al. Neutrophil response modulation activity of samples was analysed using the protocol of Tsumbu et al. Neutrophil suspension (1 million cells/143 μl PBS) was loaded in wells of 96-wells microtite plate and incubated for 10 min (at 37° C. in dark) with phospahte buffer saline solution of samples at final concentrations of 0.0125, 0.025, 0.05, 0.1 and 0.2 mg/ml. After incubation, 25 μl calcium chloride (10 μM) and 20 μl L-012 (100 μM) were added in wells. The neutrophils were activated with 10 μl PMA (16 μM) immediately before monitoring the chemiluminesence response of neutrophils during 30 min at 37° C. using Fluorosckan Ascent (Fisher Scientific, Asse, Belgium). Instead of sample dilutions only phosphate buffer saline was used in case of control.

Neutrophil response modulation activity (measured values as well as fitted curves obtained from LOESS) and Eₘₐₓ of samples are shown in FIG. 12 and FIG. 18 and table B, respectively. All the tested samples exhibited Eₘₐₓ>0%. BSF-APH, FM and CM exhibited Eₘₐₓ <40%. CM exhibited pro-oxidant behavior at 3 out of 5 tested concentrations. The IC₅₀ of samples are mentioned in table A. BSF—P and BSF-HP have the same numerical IC₅₀ values. FM: fish meal control. Methods and results are according to the methods and results in Mouithys-Mickalad et al. (2020), WO 2021/054824 and WO 2021/054823.

### Reactive Oxygen Species (ROS) Production of Macrophages

Human myeloid HL-60 cell line was purchased from American Type Culture Collection (Manassas, USA) and cultured according to the method of Boly et al. At the start of each assay: (1) cell count of suspension was estimated to maintain cellular density of 10⁶ cells/ml; (2). cell viability was measured using trypan blue assay to ensure the main viability >95% in all assays performed.

Cultured HL-60 cells were plated in Iscove's modified Dulbecco's medium. Differentiation was induced by adding 10 nM phorbol myristate acetate (PMA) dissolved in dimethyl sulfoxide (DMSO) for 24 h (37° C.). It was ensured that final concentration of DMSO in the culture medium was <0.1% and DMSO addition did not impact HL-60 proliferation, viability, and differentiation. Morphological alteration (to macrophage phenotype) in HL-60 cells resulting from differentiation was verified after 24 h of culturing using light microscopy. Post differentiation, the medium was discarded, and non-adherent cells were gently washed with Hank's balanced salt solution. Only the adherent cells were used for the assay.

Reactive oxygen species (ROS) produced by macrophages was estimated by chemi-luminescence (CL) measurement. During this assay L-012 salt (8-Amino-5-chloro-2,3-dihydro-7-phenyl-Pyrido[3,4-d]pyridazine-1,4-dione, sodium salt) was used as CL enhancer using a method adapted from Ielciu et al. Macrophages were treated with 0.05 ml of water-soluble extracts of CM, P, HP and APH to reach final concentration of 0.025, 0.05, 0.1 and 0.2 mg/ml and incubated for 1 h in the presence of 0.8 ml HBSS. Then, 20 μl of Ca²⁺ (10 mM), 20 μl of L-012 salt solution (104 M) were added prior to activation with 0.05 mL PMA and final volume was made up to 1 ml. The CL response of macrophages was monitored for 30 min at 37° C. using a Fluoroskan Ascent (Fisher Scientific, Tournai, Belgium) and expressed as the integral value of total CL emissions. For control only HBSS was added instead of water-soluble extract solution of CM, P, HP and APH.

The effect of P, HP, APH and CM on the ROS production by macrophages is displayed in FIG. 4. For P, HP and APH there was a decrease in chemi-luminescence with increasing concentration (i.e., decrease in ROS production). For CM, there was no change in chemi-luminescence with increase in concentration. Indicating that all the BSF protein derivatives and compositions reduce the ROS production, whereas CM has no effect on the ROS production by macrophages. The significant differences amongst the chemi-luminescence signals at highest concentration tested are also indicated in FIG. 4.

Macrophages have a key role in animal body due to their ability to identify, engulf and destruct pathogenic microbes or substances during phagocytosis. During OA, the activated macrophages express NOX2 genes that trigger secretion of NADH oxidase, which results in the production of ROS including superoxide anions. These ROS are further responsible for chondrocyte senescence and cartilage breakdown. The inventors no surprisingly found that all three BSF protein derivatives were able to suppress ROS production by macrophages. At highest concentration used P and APH were able to suppress ROS production >50% in comparison to control. Whereas no ROS suppression activity was observed in case of CM. Without wishing to be bound by any theory, ROS suppression activity could arise due to two mechanisms: (a). Scavenging of ROS produced by macrophage. The inventors have demonstrated the strong potential of BSF protein derivatives to scavenge ROS. In addition, the inventors also demonstrated that CM is ineffective in scavenging ROS (Mouithys-Mickalad et al., 2020). (b). Down regulation of NOX2 genes—There is well documented evidence regarding ability of specific food derived bioactive peptides to down regulate genes responsible for ROS production. Without wishing to be bound by any theory, it is possible that BSF protein derivatives also have specific peptides that could result in such down regulation. Results obtained with the here presented examples show that BSF protein derivatives can suppress ROS production from macrophages and herewith would help in prevention of OA in e.g. human subjects and pets such as dogs, cats, and in e.g. horses. Furthermore, without wishing to be bound by any theory, perhaps the BSF peptides and protein compositions have the capability to down regulate genes responsible for inflammation.

### Reactive Oxygen Species Production of PMA Activated HL-60 Cells

Human myeloid HL-60 cell line was purchased from American Type Culture Collection (Manassas, USA) and cultured according to the method of Boly et al. At the beginning of each assay: (1) cell count of suspension was estimated to maintain cellular density of 106 cells/ml; (2). cell viability was measured using trypan blue assay to main viability >95%.

Cultured HL-60 cells (5×10⁵) were suspended in 143 μl HBSS and loaded in each well of a 96-well microtiter plate and were incubated at 37° C. for 10 min with 2 μl of water-soluble extracts of CM, P, HP and APH to reach final concentration of 0.025, 0.05, 0.1 and 0.2 mg/ml. Post incubation, 20 μl of Ca²⁺ (10 mM), 20 μl of L-012 salt solution (10-4 M) were added into each well. Finally, the mixtures were activated with 10 μl PMA (16 μM). The CL measurement and control preparation was done as indicated here below in the paragraph relating to ‘Metabolic activity of HL-60 cells’. Again, chemi-luminescence was expressed as integral value of total chemi-luminescence emissions.

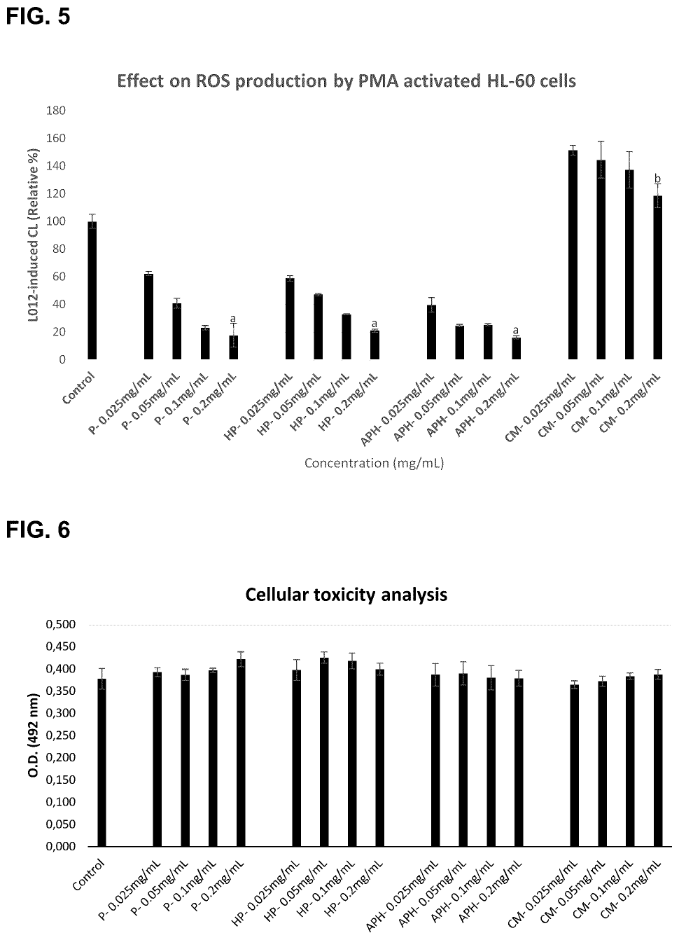

The effect of P, HP, APH and CM on the ROS production by PMA activated HL-60 cells is indicated in FIG. 5. A decrease in production of ROS was observed with increasing concentration of P, HP and APH. In case of CM, at the lowest concentration tested ROS production was 1.5 folds higher in comparison to control. The ROS production decreased with increasing concentration. However, even at the highest concentration tested CM exhibited pro-inflammatory behaviour. At final concentration % relative chemi-luminescence was in following order: CM>P=HP=APH.

Oxidative stress could trigger ROS production from monocytes which may contribute in OA development. The inventors used PMA activated HL-60 cells to mimic monocytes. Surprisingly, the inventors found that BSF protein derivatives are highly effective in suppressing the ROS production by HL-60 cells. Without wishing to be bound by any theory, the ROS suppression activity could arise due to two mechanisms: (a). Scavenging of ROS produced by the cells. The inventors have demonstrated the strong potential of BSF protein derivatives to scavenge ROS. In addition, the inventors also demonstrated that CM is ineffective in scavenging ROS (Mouithys-Mickalad et al., 2020). (b). Down regulation of NOX2 genes—There is well documented evidence regarding ability of specific food derived bioactive peptides to down regulate genes responsible for ROS production. Without wishing to be bound by any theory, it is possible that BSF protein derivatives also have specific peptides that could result in such down regulation. Results obtained with the here presented examples show that BSF protein derivatives can suppress ROS production from the cells and herewith would help in prevention of OA in e.g. human subjects and pets such as dogs. Furthermore, without wishing to be bound by any theory, perhaps the BSF peptides and protein compositions have the capability to down regulate genes responsible for inflammation. Whereas CM exhibited pro-inflammatory activity in this assay. Indicating that CM stimulates the ROS production in this assay, which may aid OA development in pets.

### Metabolic Activity of HL-60 Cells

Cultured HL-60 cells were incubated with 0.025, 0.05, 0.1 and 0.2 mg/ml of each water-soluble extract of CM, P, HP and APH for 1 h at 37° C. Post incubation, treated cells were washed two times with media and the cell metabolic activity was evaluated by adding 10 μl MTS tetrazolium salt as a cytotoxicity indicator. Absorbance of mixtures were measured after every 60 min during 2 h.

The outcomes of cellular toxicity analysis of P, HP, APH and CM are indicated in FIG. 6. None of the tested samples exhibited toxicity in this assay.

None of the tested samples had negative effect on the viability of HL-60 cells during this assay. This provides the following surprising indication: (a). BSF protein derivatives and (hydrolysed) protein compositions are not toxic to mammalian cells such as human cells and animal cells, such as cells of dogs, cats, horses; (b). ROS suppression activity seen in previous sections is not arising due to cellular mortality.

### Statistical Analysis

All the testing of the above examples was performed in triplicates. Significant differences in values obtained during glucosamine content determination, cell membrane stability, ROS production by macrophages and ROS production by PMA activated HL-60 cells analyses were examined using one-way ANOVA. Subsequently, Tukey's Range Test was performed to identify which differences were statistically significant. Differences between means were considered significantly different if p-value was less than 0.05. This analysis was conducted in GraphPad Prism 8 (GraphPad Software, San Diego, USA).

### Numbered Embodiments

Embodiment 1. Insect protein composition for use as a medicament.

Embodiment 2. Insect protein composition for use in a method for the prophylaxis or treatment of inflammation.

Embodiment 3. Insect protein composition for use in a method for the prophylaxis or treatment of inflammation in a joint, such as in a joint affected by osteoarthritis.

Embodiment 4. Insect protein composition for use in a method for the prophylaxis or treatment of synovial inflammation and/or synovitis, such as in a joint affected by osteoarthritis.

Embodiment 5. Insect protein composition for use in a method for the prophylaxis or treatment of damage of structural biomolecules of a joint, such as in a joint affected by osteoarthritis.

Embodiment 6. Insect protein composition for use in a method for the prophylaxis or treatment of chondrocyte apoptosis, such as in a joint affected by osteoarthritis.

Embodiment 7. Insect protein composition for use in a method for the prophylaxis or treatment of dysfunction of a subchondral bone and/or of subchondral bone remodeling and/or of bone loss, such as of a bone of a joint affected by osteoarthritis.

Embodiment 8. Insect protein composition for use in a method for the prophylaxis or treatment of cartilage degradation, such as of cartilage of a joint affected by osteoarthritis.

Embodiment 9. Insect protein composition for use in a method for the prophylaxis or treatment of osteophyte formation, such as in a joint affected by osteoarthritis.

Embodiment 10. Insect protein composition for use in a method for the prophylaxis or treatment of loss of articular cartilage, such as in a joint affected by osteoarthritis.

Embodiment 11. Insect protein composition for use in a method for the prophylaxis or treatment of sclerosis of subchondral bone, such as a bone of a joint affected by osteoarthritis.

Embodiment 12. Insect protein composition for use in a method for the prophylaxis or treatment of subchondral cysts formation, such as in a joint affected by osteoarthritis.

Embodiment 13. Insect protein composition for use in a method for the prophylaxis or treatment of (synovial) macrophage activation and/or (activated) (synovial) macrophage-induced synovial damage, such as in synovium of a joint affected by osteoarthritis.

Embodiment 14. Insect protein composition for use in a method for the prophylaxis or treatment of low-grade inflammatory disease of a joint, such as of a joint affected by osteoarthritis.

Embodiment 15. Insect protein composition for use in a method for the prophylaxis or treatment of joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis.

Embodiment 16. Insect protein composition for use in a method for the prophylaxis or treatment of innate immune response in a joint, such as in a joint affected by osteoarthritis.

Embodiment 17. Insect protein composition for use in a method for the prophylaxis or treatment of joint stiffness and/or locomotor restriction and/or flexion contracture and/or decreased flexion, such as of a joint affected by osteoarthritis.

Embodiment 18. Insect protein composition for use in a method for the prophylaxis or treatment of synovial fibrosis, such as in a joint affected by osteoarthritis.

Embodiment 19. Insect protein composition for use in a method for the prophylaxis or treatment of joint-space narrowing and/or cartilage degradation in a joint, such as in a joint affected by osteoarthritis.

Embodiment 20. Insect protein composition for use in a method for the prophylaxis or treatment of deterioration of articular cartilage and underlying bone, such as in a joint affected by osteoarthritis.

Embodiment 21. Insect protein composition for use in a method for the prophylaxis or treatment of erythrocyte lysis and/or elevated erythrocyte sedimentation compared to normal, such as erythrocyte lysis and/or elevated erythrocyte sedimentation in synovium of a joint affected by osteoarthritis.

Embodiment 22. Insect protein composition for use in a method for the prophylaxis or treatment of arthritis.

Embodiment 23. Insect protein composition for use in a method for the prophylaxis or treatment of osteoarthritis.

Embodiment 24. Insect protein composition for use in a method for the prophylaxis or treatment of osteoarthritis progression.

Embodiment 25. Insect protein composition for use in a method for the prophylaxis or treatment of erosive osteoarthritis or inflammatory osteoarthritis.

Embodiment 26. Insect protein composition for use in a method for the prophylaxis or treatment of early-stage osteoarthritis or late-stage osteoarthritis.

Embodiment 27. Insect protein composition for use according to any one of the embodiments 1-26, wherein the insect is black soldier fly.

Embodiment 28. Insect protein composition for use according to any one of the embodiments 1-26, wherein the insect is larvae of black soldier fly.

Embodiment 29. Insect protein composition for use according to any one of the embodiments 1-28, wherein the insect protein composition is minced and heated insects, preferably black soldier fly, more preferably larvae of black soldier fly.

Embodiment 30. Insect protein composition for use according to any one of the embodiments 1-29, wherein the insect protein composition is enzymatically hydrolyzed minced and heated insects, preferably black soldier fly, more preferably larvae of black soldier fly.

Embodiment 31. Insect protein composition for use according to any one of the embodiments 1-30, wherein the insect protein composition is the water-soluble extract of minced and heated insects, preferably black soldier fly, more preferably larvae of black soldier fly, or the water-soluble extract of enzymatically hydrolyzed minced and heated insects, preferably black soldier fly, more preferably larvae of black soldier fly.

Embodiment 32. Insect protein composition for use according to any one of the embodiments 1-30, wherein the insect protein composition is the enzymatically hydrolyzed water-soluble extract of minced and heated insects, preferably black soldier fly, more preferably larvae of black soldier fly.

Embodiment 33. Insect protein composition for use according to any one of the embodiments 1-28, wherein the insect protein composition is heated enzymatically hydrolyzed minced insects, preferably black soldier fly, more preferably larvae of black soldier fly.

Embodiment 34. Insect protein composition for use according to any one of the embodiments 1-28, wherein the insect protein composition is the water-soluble extract of heated enzymatically hydrolyzed minced insects, preferably black soldier fly, more preferably larvae of black soldier fly.

Embodiment 35. Insect protein composition for use according to any one of the embodiments 1-34, wherein the insect protein composition is orally administered.

Embodiment 36. Insect protein composition for use according to any one of the embodiments 1-35, wherein the insect protein composition is administered to a mammal.

Embodiment 37. Insect protein composition for use according to any one of the embodiments 1-36, wherein the insect protein composition is administered to a human subject or to a human patient, such as a human patient suffering from osteoarthritis.

Embodiment 38. Insect protein composition for use according to any one of the embodiments 1-35, wherein the insect protein composition is administered to an animal.

Embodiment 39. Insect protein composition for use according to any one of the embodiments 1-35, wherein the insect protein composition is administered to a horse, a pet such as a dog or a cat, such as a horse, a pet such as a dog or a cat suffering from osteoarthritis.

Embodiment 40. Insect protein composition for use according to any one of the embodiments 1-39, wherein the insect protein composition is administered to a human patient suffering from any one or more of: inflammation, for example inflammation in a joint, such as in a joint affected by osteoarthritis, synovial inflammation and/or synovitis, such as in a joint affected by osteoarthritis, damage of structural biomolecules of a joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation and/or macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis.

Embodiment 41. Insect protein composition for use according to any one of the embodiments 1-40, wherein the insect protein composition has any one or more of the following activities:
(a) inhibiting macrophage activation and/or preventing macrophage activation;
(b) preventing and/or inhibiting cell lysis such as erythrocyte lysis; and
(c) inhibiting and/or preventing reactive oxygen species formation by a cell such as an endothelial cell or a macrophage, and/or comprises glucosamine and/or glucosamine-sulphate. The amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

Embodiment 42. Insect protein composition for use according to any one of the embodiments 1-41, wherein the insect protein composition inhibits and/or prevents macrophage activation and/or prevents or inhibits ROS formation by a(n) (activated) macrophage; prevents and/or inhibits reactive oxygen species formation by a cell such as an endothelial cell or a macrophage; and comprises glucosamine. The amount of glucosamine is typically 0.05-5 wt % glucosamine based on the dry weight of the insect protein composition, preferably 0.1-2.5 wt %, more preferably 0.2-1.25 wt %, such as 0.25-1.0 wt %.

Embodiment 43. Human food supplement or animal feed supplement, human food ingredient or animal feed ingredient or human food product or animal feed product, comprising the insect protein composition according to any one of the embodiments 27-34, 41, 42.

Embodiment 44. Use of the insect protein composition according to any one of the embodiments 27-34, 41, 42 in the preparation of a human food supplement or animal feed supplement, human food ingredient or animal feed ingredient or human food product or animal feed product.

Embodiment 45. Animal feed supplement, ingredient or product of embodiment 43 or use of the insect protein composition in the preparation of an animal feed supplement, ingredient or product according to embodiment 44, wherein the animal is a horse or a pet such as a dog or a cat.

Embodiment 46. Non-therapeutic method of prevention against, providing relief from or amelioration of a joint problem in a human subject or an animal such as a horse, a pet such as a dog or a cat, preferably a human subject or a dog, the method comprising orally administering to the human subject or the animal the insect protein composition of any one of the embodiments 27-34, 41, 42 or the human food supplement or animal feed supplement, human food ingredient or animal feed ingredient or human food product or animal feed product of embodiment 43 or 45,
wherein the joint problem is any one or more of:
inflammation, for example inflammation in a joint affected by osteoarthritis, synovial inflammation and/or synovitis, such as in a joint affected by osteoarthritis, damage of structural biomolecules of the joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation and/or macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis.

Embodiment 47. Non-therapeutic method of prevention against, providing relief from or amelioration of a joint problem in a human subject or an animal such as a horse, a pet such as a dog or a cat, preferably a human subject or a dog according to embodiment 46, wherein the joint problem is osteoarthritis and wherein the human subject is a human subject not suffering from osteoarthritis or is a human patient suffering from osteoarthritis, and wherein the animal such as a horse, dog or cat, preferably a dog, is a dog not suffering from osteoarthritis or is a dog suffering from osteoarthritis.

Embodiment 48. Pharmaceutical composition comprising the insect protein composition of any one of the embodiments 27-34, 41, 42 and optionally a pharmaceutically acceptable diluent and/or a pharmaceutically acceptable excipient.

Embodiment 49. Pharmaceutical composition of embodiment 48, formulated for oral administration.

Embodiment 50. Pharmaceutical composition of embodiment 48 or 49 for use according to any one of the embodiments 1-26.

Embodiment 51. Pharmaceutical composition for use of embodiment 50 in a method of treatment or prophylaxis of any one or more of osteoarthritis, osteoarthritis progression, erosive osteoarthritis, inflammatory osteoarthritis, early-stage osteoarthritis or late-stage osteoarthritis, wherein a human subject is treated or an animal such as a horse or a pet, for example a dog or a cat, preferably a human patient or a dog, such as a human patient or a dog suffering from osteoarthritis.

Embodiment 52. A method for the prevention or treatment of inflammation in a subject in need thereof, the method comprising administering an insect protein composition to the subject.

Embodiment 53. A method for the prevention or treatment of inflammation in a joint, such as in a joint affected by osteoarthritis, in a subject in need thereof, the method comprising administering an insect protein composition to the subject.

Embodiment 54. A method for the prevention or treatment of synovial inflammation and/or synovitis, such as in a joint affected by osteoarthritis, in a subject in need thereof, the method comprising administering an insect protein composition to the subject.

Embodiment 55. A method for the prevention or treatment of structural biomolecules of a joint, such as in a joint affected by osteoarthritis, in a subject in need thereof, the method comprising administering an insect protein composition to the subject.

Embodiment 56. A method for the prevention or treatment of chondrocyte apoptosis, such as in a joint affected by osteoarthritis, in a subject in need thereof, the method comprising administering an insect protein composition to the subject.

Embodiment 57. A method for the prevention or treatment of dysfunction of a subchondral bone and/or of subchondral bone remodeling and/or of bone loss, such as of a bone of a joint affected by osteoarthritis, in a subject in need thereof, the method comprising administering an insect protein composition to the subject.

Embodiment 58. A method for the prevention or treatment of cartilage degradation, such as of cartilage of a joint affected by osteoarthritis, in a subject in need thereof, the method comprising administering an insect protein composition to the subject.

Embodiment 59. A method for the prevention or treatment of osteophyte formation, such as in a joint affected by osteoarthritis, in a subject in need thereof, the method comprising administering an insect protein composition to the subject.

Embodiment 60. A method for the prevention or treatment of loss of articular cartilage, such as in a joint affected by osteoarthritis, in a subject in need thereof, the method comprising administering an insect protein composition to the subject.

Embodiment 61. A method for the prevention or treatment of sclerosis of subchondral bone, such as a bone of a joint affected by osteoarthritis, in a subject in need thereof, the method comprising administering an insect protein composition to the subject.

Embodiment 62. A method for the prevention or treatment of subchondral cysts formation, such as in a joint affected by osteoarthritis, in a subject in need thereof, the method comprising administering an insect protein composition to the subject.

Embodiment 63. A method for the prevention or treatment of synovial macrophage activation and/or the prevention or treatment of macrophage-induced synovial damage, such as in synovium of a joint affected by osteoarthritis, in a subject in need thereof, the method comprising administering an insect protein composition to the subject.

Embodiment 64. A method for the prevention or treatment of low-grade inflammatory disease of a joint, such as of a joint affected by osteoarthritis, in a subject in need thereof, the method comprising administering an insect protein composition to the subject.

Embodiment 65. A method for the prevention or treatment of joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, in a subject in need thereof, the method comprising administering an insect protein composition to the subject.

Embodiment 66. A method for the prevention or treatment of innate immune response in a joint, such as in a joint affected by osteoarthritis, in a subject in need thereof, the method comprising administering an insect protein composition to the subject.

Embodiment 67. A method for the prevention or treatment of joint stiffness and/or locomotor restriction and/or flexion contracture and/or decreased flexion, such as of a joint affected by osteoarthritis, in a subject in need thereof, the method comprising administering an insect protein composition to the subject.

Embodiment 68. A method for the prevention or treatment of synovial fibrosis, such as in a joint affected by osteoarthritis, in a subject in need thereof, the method comprising administering an insect protein composition to the subject.

Embodiment 69. A method for the prevention or treatment of joint-space narrowing, such as in a joint affected by osteoarthritis, in a subject in need thereof, the method comprising administering an insect protein composition to the subject.

Embodiment 70. A method for the prevention or treatment of deterioration of articular cartilage and underlying bone, such as in a joint affected by osteoarthritis, in a subject in need thereof, the method comprising administering an insect protein composition to the subject.

Embodiment 71. A method for the prevention or treatment of erythrocyte lysis and/or elevated erythrocyte sedimentation compared to normal, such as erythrocyte lysis and/or elevated erythrocyte sedimentation in synovium of a joint affected by osteoarthritis, in a subject in need thereof, the method comprising administering an insect protein composition to the subject.

Embodiment 72. A method for the prevention or treatment of arthritis in a subject in need thereof, the method comprising administering an insect protein composition to the subject.

Embodiment 73. A method for the prevention or treatment of osteoarthritis in a subject in need thereof, the method comprising administering an insect protein composition to the subject.

Embodiment 74. A method for the prevention or treatment of osteoarthritis progression in a subject in need thereof, the method in a subject in need thereof, comprising administering an insect protein composition to the subject.

Embodiment 75. A method for the prevention or treatment of erosive osteoarthritis or inflammatory osteoarthritis, in a subject in need thereof, comprising administering an insect protein composition to the subject.

Embodiment 76. A method for the prevention or treatment of early-stage osteoarthritis or late-stage osteoarthritis, in a subject in need thereof, comprising administering an insect protein composition to the subject.

Embodiment 77. The method of any one of embodiments 52-76, wherein the insect is black soldier fly.

Embodiment 78. The method of any one of embodiments 52-76, wherein the insect is larvae of black soldier fly.

Embodiment 79. The method of any one of embodiments 52-78, wherein the insect protein composition is minced and heated insects, preferably black soldier fly, more preferably larvae of black soldier fly.

Embodiment 80. The method of any one of embodiments 52-79, wherein the insect protein composition is enzymatically hydrolyzed minced and heated insects, preferably black soldier fly, more preferably larvae of black soldier fly.

Embodiment 81. The method of any one of embodiments 52-80, wherein the insect protein composition is the water-soluble extract of minced and heated insects, preferably black soldier fly, more preferably larvae of black soldier fly, or the water-soluble extract of enzymatically hydrolyzed minced and heated insects, preferably black soldier fly, more preferably larvae of black soldier fly.

Embodiment 82. The method of any one of embodiments 52-80, wherein the insect protein composition is the enzymatically hydrolyzed water-soluble extract of minced and heated insects, preferably black soldier fly, more preferably larvae of black soldier fly.

Embodiment 83. The method of any one of embodiments 52-78, wherein the insect protein composition is heated enzymatically hydrolyzed minced insects, preferably black soldier fly, more preferably larvae of black soldier fly.

Embodiment 84. The method of any one of embodiments 52-78, wherein the insect protein composition is the water-soluble extract of heated enzymatically hydrolyzed minced insects, preferably black soldier fly, more preferably larvae of black soldier fly.

Embodiment 85. The method of any one of embodiments 52-84, wherein the insect protein composition is orally administered.

Embodiment 86. The method of any one of embodiments 52-85, wherein the subject is a mammal.

Embodiment 87. The method of any one of embodiments 52-86, wherein the subject in need thereof is a human subject or to a human patient, such as a human patient suffering from osteoarthritis.

Embodiment 88. The method of any one of embodiments 52-85, wherein the subject is an animal.

Embodiment 89. The method of any one of embodiments 52-85, wherein the subject is a horse, a pet such as a dog or a cat, such as a horse, a pet such as a dog or a cat suffering from osteoarthritis.

Embodiment 90. The method of any one of embodiments 52-89, wherein the subject in need thereof is a human patient suffering from any one or more of: inflammation, for example inflammation in a joint, such as in a joint affected by osteoarthritis, synovial inflammation and/or synovitis, such as in a joint affected by osteoarthritis, damage of structural biomolecules of a joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation and/or macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis.

Embodiment 91. The method of any one of embodiments 52-90, wherein the insect protein composition has any one or more of the following activities:
(a) inhibiting macrophage activation and/or preventing macrophage activation, and/or preventing or inhibiting macrophage-induced synovial damage;
(b) preventing and/or inhibiting cell lysis such as erythrocyte lysis; and
(c) inhibiting and/or preventing reactive oxygen species formation by a cell such as an endothelial cell or a macrophage, and/or comprises glucosamine and/or glucosamine-sulphate.

Embodiment 92. The method of any one of embodiments 52-91, wherein the insect protein composition inhibits and/or prevents macrophage activation and/or prevents or inhibits macrophage-induced synovial damage; prevents and/or inhibits reactive oxygen species formation by a cell such as an endothelial cell or a macrophage; and comprises glucosamine.

Embodiment 93. Human food supplement or animal feed supplement, human food ingredient or animal feed ingredient or human food product or animal feed product, comprising the insect protein composition of any one of embodiments 77-84, 92, 92.

Embodiment 94. Animal feed supplement, ingredient or product of embodiment 43, wherein the animal is a horse or a pet such as a dog or a cat.

Embodiment 95. Pharmaceutical composition comprising the insect protein composition of any one of embodiments 77-84, 91, 92 and optionally a pharmaceutically acceptable diluent and/or a pharmaceutically acceptable excipient.

Embodiment 96. Pharmaceutical composition of embodiment 95 formulated for oral administration.

Embodiment 97. A method for the prevention or treatment of any one or more of osteoarthritis, osteoarthritis progression, erosive osteoarthritis, inflammatory osteoarthritis, early-stage osteoarthritis or late-stage osteoarthritis, in a subject in need thereof, the method comprising administering the pharmaceutica composition of embodiment 95 or 96, wherein the subject is human or an animal such as a horse or a pet, for example a dog or a cat, preferably a human patient or a dog, such as a human patient or a dog suffering from osteoarthritis.

### REFERENCES

Arnao, M. B.; Cano, A.; Acosta, M. The hydrophilic and lipophilic contribution to total antioxidant activity. Food Chemistry 2001, 73, 239-244, doi:10.1016/S0308-8146(00)00324-1.
Brand-Williams, W.; Cuvelier, M. E.; Berset, C. Use of a free radical method to evaluate antioxidant activity. LWT—Food Science and Technology 1995, 28, 25-30, doi:10.1016/S0023-6438(95)80008-5.
Rainatou Boly, Thierry Franck, Stephan Kohnen, Marius Lompo, Innocent Pierre Guissou, Jacques Dubois, Didier Serteyn, and AngeMouithys-Mickalad, Evaluation of Antiradical and Anti-Inflammatory Activities of Ethyl Acetate and Butanolic Subfractions of Agelanthus dodoneifolius (DC.) Polhill & Wiens (Loranthaceae) Using Equine Myeloperoxidase and Both PMA-Activated Neutrophils and HL-60 Cells, Evidence-Based Complementary and Alternative Medicine Volume 2015, Article ID 707524, 9 pages, 1-9, dx.doi.org/10.1155/2015/707524
Franck, T.; Kohnen, S.; Boudjeltia, K. Z.; Van Antwerpen, P.; Bosseloir, A.; Niesten, A.; Gach, O.; Nys, M.; Deby-Dupont, G.; Serteyn, D. A new easy method for specific measurement of active myeloperoxidase in human biological fluids and tissue extracts. Talanta 2009, 80, 723-729, doi:10.1016/j.talanta.2009.07.052.
Ielciu, I.; Mouithys-Mickalad, A.; Franck, T.; Angenot, L.; Ledoux, A.; P\check{a}ltinean, R.; Cieckiewicz, E.; Etienne, D.; Tits, M.; Crişan, G.; et al. Flavonoid Composition, Cellular Antioxidant Activity and (Myelo) Peroxidase Inhibition of a Bryonia Alba L. (Cucurbitaceae) Leaves Extract. J. Pharm. Pharmacol. 2019, 71, 230-239, doi:10.1111/jphp.13025. PMID: 30324727
Gholamreza Karimi, Mohammad Hassanzadeh, Soghra Mehri, Protective Effect of Rosmarinus officinalis L. Essential Oil against Free Radical-Induced Erythrocyte Lysis, Iranian Journal of Pharmaceutical Sciences, Autumn 2005: 1(4): 231-236
Loeser, R. F., Goldring, S. R., Scanzello, C. R., Goldring, M. B., 2012. Osteoarthritis: A Disease of the Joint as an Organ. Arthritis Rheum. 64, 1697-1707. doi.org/10.1002/art.34453
Ange Mouithys-Mickalad, Eric Schmitt, Monika Dalim, Thierry Franck, Nuria Martin Tome, Michel van Spankeren, Didier Serteyn and Aman Paul, Black Soldier Fly (Hermetia illucens) Larvae Protein Derivatives: Potential to Promote Animal Health, Animals 2020, 10, 941; doi:10.3390/ani10060941
Selvakumar Murugesan, Meenakshi R. Venkateswaran, Sasidharan Jayabal, Sureshkumar Periyasamy, Evaluation of the antioxidant and anti-arthritic potential of Zingiber officinale Rosc. by in vitro and in silico analysis, South African Journal of Botany 130, pp. 45-53, 2020, doi.org/10.1016/j.sajb.2019.12.019
Paul, A. Field border flowering strips as a source of valuable compounds, Gembloux Agro-Bio Tech University of Liège, Gembloux, Belgique, 2017.
Carol Robertson-Plouch, John R. Stille, Peng Liu, Claire Smith, Dorothy Brown, Margaret Warner, Leijun Hu, Matthew J. Fisher, OSTEOARTHRITIS—A randomized clinical efficacy study targeting mPGES1 or EP4 in dogs with spontaneous osteoarthritis, Sci. Transl. Med. 11, eaaw9993 (2019) 30 Oct. 2019, page 1-12
Smith, P. K.; Krohn, R. I.; Hermanson, G. T.; Mallia, A. K.; Gartner, F. H.; Provenzano, M. D.; Fujimoto, E. K.; Goeke, N. M.; Olson, B. J.; Klenk, D. C. Measurement of protein using bicinchoninic acid. Anal. Biochem. 1985, 150, 76-85, doi:10.1016/0003-2697(85)90442-7.
Tsumbu, C. N.; Deby-Dupont, G.; Tits, M.; Angenot, L.; Frederich, M.; Kohnen, S.; Mouithys-Mickalad, A.; Serteyn, D.; Franck, T. Polyphenol content and modulatory activities of some tropical dietary plant extracts on the oxidant activities of neutrophils and myeloperoxidase. Int J Mol Sci 2012, 13, 628-650, doi:10.3390/ijms13010628.

1. Insect protein composition for use as a medicament.

2. Insect protein composition for use in a method for the prophylaxis or treatment of inflammation.

3. Insect protein composition for use in a method for the prophylaxis or treatment of inflammation in a joint, such as in a joint affected by osteoarthritis.

4. Insect protein composition for use in a method for the prophylaxis or treatment of synovial inflammation and/or synovitis, such as in a joint affected by osteoarthritis.

5. Insect protein composition for use in a method for the prophylaxis or treatment of damage of structural biomolecules of a joint, such as in a joint affected by osteoarthritis.

6. Insect protein composition for use in a method for the prophylaxis or treatment of chondrocyte apoptosis, such as in a joint affected by osteoarthritis.

7. Insect protein composition for use in a method for the prophylaxis or treatment of dysfunction of a subchondral bone and/or of subchondral bone remodeling and/or of bone loss, such as of a bone of a joint affected by osteoarthritis.

8. Insect protein composition for use in a method for the prophylaxis or treatment of cartilage degradation, such as of cartilage of a joint affected by osteoarthritis.

9. Insect protein composition for use in a method for the prophylaxis or treatment of osteophyte formation, such as in a joint affected by osteoarthritis.

10. Insect protein composition for use in a method for the prophylaxis or treatment of loss of articular cartilage, such as in a joint affected by osteoarthritis.

11. Insect protein composition for use in a method for the prophylaxis or treatment of sclerosis of subchondral bone, such as a bone of a joint affected by osteoarthritis.

12. Insect protein composition for use in a method for the prophylaxis or treatment of subchondral cysts formation, such as in a joint affected by osteoarthritis.

13. Insect protein composition for use in a method for the prophylaxis or treatment of synovial macrophage activation and/or the prevention or treatment of macrophage-induced synovial damage, such as in synovium of a joint affected by osteoarthritis.

14. Insect protein composition for use in a method for the prophylaxis or treatment of low-grade inflammatory disease of a joint, such as of a joint affected by osteoarthritis.

15. Insect protein composition for use in a method for the prophylaxis or treatment of joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis.

16. Insect protein composition for use in a method for the prophylaxis or treatment of innate immune response in a joint, such as in a joint affected by osteoarthritis.

17. Insect protein composition for use in a method for the prophylaxis or treatment of joint stiffness and/or locomotor restriction and/or flexion contracture and/or decreased flexion, such as of a joint affected by osteoarthritis.

18. Insect protein composition for use in a method for the prophylaxis or treatment of synovial fibrosis, such as in a joint affected by osteoarthritis.

19. Insect protein composition for use in a method for the prophylaxis or treatment of joint-space narrowing, such as in a joint affected by osteoarthritis.

20. Insect protein composition for use in a method for the prophylaxis or treatment of deterioration of articular cartilage and underlying bone, such as in a joint affected by osteoarthritis.

21. Insect protein composition for use in a method for the prophylaxis or treatment of erythrocyte lysis and/or elevated erythrocyte sedimentation compared to normal, such as erythrocyte lysis and/or elevated erythrocyte sedimentation in synovium of a joint affected by osteoarthritis.

22. Insect protein composition for use in a method for the prophylaxis or treatment of arthritis.

23. Insect protein composition for use in a method for the prophylaxis or treatment of osteoarthritis.

24. Insect protein composition for use in a method for the prophylaxis or treatment of osteoarthritis progression.

25. Insect protein composition for use in a method for the prophylaxis or treatment of erosive osteoarthritis or inflammatory osteoarthritis.

26. Insect protein composition for use in a method for the prophylaxis or treatment of early-stage osteoarthritis or late-stage osteoarthritis.

27. Insect protein composition for use according to any one of the claims 1-26, wherein the insect is black soldier fly.

28. Insect protein composition for use according to any one of the claims 1-26, wherein the insect is larvae of black soldier fly.

29. Insect protein composition for use according to any one of the claims 1-28, wherein the insect protein composition is minced and heated insects, preferably black soldier fly, more preferably larvae of black soldier fly.

30. Insect protein composition for use according to any one of the claims 1-29, wherein the insect protein composition is enzymatically hydrolyzed minced and heated insects, preferably black soldier fly, more preferably larvae of black soldier fly.

31. Insect protein composition for use according to any one of the claims 1-30, wherein the insect protein composition is the water-soluble extract of minced and heated insects, preferably black soldier fly, more preferably larvae of black soldier fly, or the water-soluble extract of enzymatically hydrolyzed minced and heated insects, preferably black soldier fly, more preferably larvae of black soldier fly.

32. Insect protein composition for use according to any one of the claims 1-30, wherein the insect protein composition is the enzymatically hydrolyzed water-soluble extract of minced and heated insects, preferably black soldier fly, more preferably larvae of black soldier fly.

33. Insect protein composition for use according to any one of the claims 1-28, wherein the insect protein composition is heated enzymatically hydrolyzed minced insects, preferably black soldier fly, more preferably larvae of black soldier fly.

34. Insect protein composition for use according to any one of the claims 1-28, wherein the insect protein composition is the water-soluble extract of heated enzymatically hydrolyzed minced insects, preferably black soldier fly, more preferably larvae of black soldier fly.

35. Insect protein composition for use according to any one of the claims 1-34, wherein the insect protein composition is orally administered.

36. Insect protein composition for use according to any one of the claims 1-35, wherein the insect protein composition is administered to a mammal.

37. Insect protein composition for use according to any one of the claims 1-36, wherein the insect protein composition is administered to a human subject or to a human patient, such as a human patient suffering from osteoarthritis.

38. Insect protein composition for use according to any one of the claims 1-35, wherein the insect protein composition is administered to an animal.

39. Insect protein composition for use according to any one of the claims 1-35, wherein the insect protein composition is administered to a horse, a pet such as a dog or a cat, such as a horse, a pet such as a dog or a cat suffering from osteoarthritis.

40. Insect protein composition for use according to any one of the claims 1-39, wherein the insect protein composition is administered to a human patient suffering from any one or more of: inflammation, for example inflammation in a joint, such as in a joint affected by osteoarthritis, synovial inflammation and/or synovitis, such as in a joint affected by osteoarthritis, damage of structural biomolecules of a joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation and/or macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis.

41. Insect protein composition for use according to any one of the claims 1-40, wherein the insect protein composition has any one or more of the following activities:
(a) inhibiting macrophage activation and/or preventing macrophage activation, and/or preventing or inhibiting macrophage-induced synovial damage;
(b) preventing and/or inhibiting cell lysis such as erythrocyte lysis; and
(c) inhibiting and/or preventing reactive oxygen species formation by a cell such as an endothelial cell or a macrophage,
and/or comprises glucosamine and/or glucosamine-sulphate.

42. Insect protein composition for use according to any one of the claims 1-41, wherein the insect protein composition inhibits and/or prevents macrophage activation and/or prevents or inhibits macrophage-induced synovial damage; prevents and/or inhibits reactive oxygen species formation by a cell such as an endothelial cell or a macrophage; and comprises glucosamine.

43. Human food supplement or animal feed supplement, human food ingredient or animal feed ingredient or human food product or animal feed product, comprising the insect protein composition according to any one of the claims 27-34, 41, 42.

44. Use of the insect protein composition according to any one of the claims 27-34, 41, 42 in the preparation of a human food supplement or animal feed supplement, human food ingredient or animal feed ingredient or human food product or animal feed product.

45. Animal feed supplement, ingredient or product of claim 43 or use of the insect protein composition in the preparation of an animal feed supplement, ingredient or product according to claim 44, wherein the animal is a horse or a pet such as a dog or a cat.

46. Non-therapeutic method of prevention against, providing relief from or amelioration of a joint problem in a human subject or an animal such as a horse, a pet such as a dog or a cat, preferably a human subject or a dog,
the method comprising orally administering to the human subject or the animal the insect protein composition of any one of the claims 27-34, 41, 42 or the human food supplement or animal feed supplement, human food ingredient or animal feed ingredient or human food product or animal feed product of claim 43 or 45,
wherein the joint problem is any one or more of:
inflammation, for example inflammation in a joint affected by osteoarthritis, synovial inflammation and/or synovitis, such as in a joint affected by osteoarthritis, damage of structural biomolecules of the joint, such as in a joint affected by osteoarthritis, chondrocyte apoptosis, such as in a joint affected by osteoarthritis, dysfunction of a subchondral bone and/or of subchondral bone remodeling and/or of bone loss, such as of a bone of a joint affected by osteoarthritis, cartilage degradation, such as of cartilage of a joint affected by osteoarthritis, osteophyte formation, such as in a joint affected by osteoarthritis, loss of articular cartilage, such as in a joint affected by osteoarthritis, sclerosis of subchondral bone, such as a bone of a joint affected by osteoarthritis, subchondral cysts formation, such as in a joint affected by osteoarthritis, synovial macrophage activation and/or macrophage-induced synovial damage, such as in synovium of a joint affected by osteoarthritis, low-grade inflammatory disease of a joint, such as of a joint affected by osteoarthritis, joint pain, such as immobilizing joint pain, and/or of joint swelling and/or of joint tenderness and/or of knee-joint patellar ballottement and/or of joint effusion, such as knee joint effusion, such as relating to a joint affected by osteoarthritis, innate immune response in a joint, such as in a joint affected by osteoarthritis, joint stiffness and/or locomotor restriction and/or flexion contracture and/or decreased flexion, such as of a joint affected by osteoarthritis, synovial fibrosis, such as in a joint affected by osteoarthritis, joint-space narrowing, such as in a joint affected by osteoarthritis, deterioration of articular cartilage and underlying bone, such as in a joint affected by osteoarthritis, erythrocyte lysis and/or elevated erythrocyte sedimentation compared to normal, such as erythrocyte lysis and/or elevated erythrocyte sedimentation in synovium of a joint affected by osteoarthritis, arthritis, osteoarthritis, osteoarthritis progression, erosive osteoarthritis or inflammatory osteoarthritis, and early-stage osteoarthritis or late-stage osteoarthritis.

47. Non-therapeutic method of prevention against, providing relief from or amelioration of a joint problem in a human subject or an animal such as a horse, a pet such as a dog or a cat, preferably a human subject or a dog according to claim 46, wherein the joint problem is osteoarthritis and wherein the human subject is a human subject not suffering from osteoarthritis or is a human patient suffering from osteoarthritis, and wherein the animal such as a horse, dog or cat, preferably a dog, is a dog not suffering from osteoarthritis or is a dog suffering from osteoarthritis.

48. Pharmaceutical composition comprising the insect protein composition of any one of the claims 27-34, 41, 42 and optionally a pharmaceutically acceptable diluent and/or a pharmaceutically acceptable excipient.

49. Pharmaceutical composition of claim 48, formulated for oral administration.

50. Pharmaceutical composition of claim 48 or 49 for use according to any one of the claims 1-26.

51. Pharmaceutical composition for use of claim 50 in a method of treatment or prophylaxis of any one or more of osteoarthritis, osteoarthritis progression, erosive osteoarthritis, inflammatory osteoarthritis, early-stage osteoarthritis or late-stage osteoarthritis, wherein a human subject is treated or an animal such as a horse or a pet, for example a dog or a cat, preferably a human patient or a dog, such as a human patient or a dog suffering from osteoarthritis.
